(19) United States
(12) Patent Application Publication
(10) Pub. No.: US 2025/0206698 A1
(43) Pub. Date: Jun. 26, 2025

(54) Process for Making Lisdexamfetamine Dimesylate

(71) Applicant: PHARMAPOTHECA A, INC., DEL MAR, NY (US)

(72) Inventors: HAROLD MECKLER, DELMAR, NY (US); DARRYL CLEARY, CHAPEL HILL, NC (US); MARCUS BRACKEEN, DURHAM, NC (US); SREENIVASULU KODURU, WILMINGTON, NC (US); NILAMBER MATE, WILMINGTON, NC (US); KARL POPP, DUCK POND, NY (US)

(21) Appl. No.: 19/000,607

(22) Filed: Dec. 23, 2024

Related U.S. Application Data
(60) Provisional application No. 63/614,450, filed on Dec. 22, 2023.

(51) Int. Cl. C07C 231/14; C07C 231/22

(52) U.S. Cl. CPC C07C 231/14; C07C 231/22

(57) ABSTRACT

The invention relates to a novel process for making lisdexamfetamine dimesylate and related derivatives by the novel amino acid activation and amphetamine acylation in the presence of water or alcohols. A second invention relates to the novel process for making lisdexamfetamine dimesylate from racemic amphetamine compounds where the single isomer product is isolated without a chemical or enzymatic resolution.

### BACKGROUND

### Field of the Invention

The invention relates to chemical processes for making a pharmaceutical, and in particular, to a process for making lisdexamfetamine dimesylate.

### Background of the Art

Lisdexamfetamine dimesylate (LDX), is chemically known as (2S)-2,6-diamino-N-[(1S)-1-methyl-2-phenylethyl]hexanamide dimethanesulfonate, and is a long-acting, central nervous system (CNS) stimulating drug with low toxicity used as an abuse-resistant treatment of attention-deficit/hyperactivity disorder (ADHD). LDX is a therapeutically inactive amphetamine prodrug, and the pharmacologically active D-amphetamine is gradually released by rate-limited hydrolysis following ingestion. The drug was invented and developed by New River Pharmaceuticals, Inc. (Radford, VA, USA) and was globally commercialized by Shire PLC. (Jersey, UK). LDX is currently marketed under the trade name of Vyvanse since its launch in February 2007. LDX was patented in the U.S. as U.S. Pat. Nos. 7,655,630, 7,659,253, and 7,662,787.

The original process to LDX had a problem of producing dicyclohexylurea as a process by-product. U.S. Pat. No. 11,608,312 replaced DCC with EDCI to address the problem and remove dicyclohexylurea as a process by-product.

A one-pot process to LDX was patented by SpecGX LLC as U.S. Pat. No. 10,927,068.

Archimica, now Curia, patented another process to LDX in U.S. Pat. No. 8,487,134 using protected lysine with dexamphetamine in propanephosphonic acid (T3P) in ethyl acetate, followed by deprotection in 1,4-dioxane and methanesulfonic acid.

Cambrex patented another process to LDX in U.S. Pat. No. 8,614,346 using a Vilsmeier reagent.

Noramco patented an enzymatic process to LDX in U.S. Pat. No. 10,544,434.

Orchid Chemicals disclosed a process to LDX in IN2011/DE02040.

Sun Pharmaceuticals disclosed a process to LDX in WO2017/098533 A2.

However, improved manufacturing processes are still needed within the field.

### SUMMARY OF THE INVENTION

The invention relates to a novel process for making lisdexamfetamine dimesylate.

In a preferred embodiment, the invention comprises (i) reacting a protected L-Lysine ammonium salt BOC-L-Lys(BOC)—O—X—NH+ such as Et₃NH+ in an organic solvent with an amphetamine in aqueous base (dextroamphetamine, levoamphetamine or the racemic mixture) in the presence of a coupling agent such as DMTMM ((4-(4,6-dimethoxy-1,3,5-triazin-2-yl)-4-methyl-morpholinium chloride) to form a biphasic mixture and heating the biphasic mixture at 30-50° C., preferably 40-50° C., to couple the amphetamine (dextroamphetamine or levoamphetamine or the mixture) with the protected lysine carboxylic acid and yield respective the amides BOC-L-Lys(BOC)—N-dextroamphetamine (or BOC-L-Lys(BOC)—N-levoamphetamine or the racemic mixture), and then (ii) reacting the BOC-L-Lys(BOC)—N-dextroamphetamine (or levoamphetamine or the racemic mixture) with methanesulfonic acid (MsOH) in isopropanol and isopropyl acetate, and crystallizing the product to obtain lisdexamfetamine dimesylate, lislevoamfetamine dimesylate or a racemic mixture of lisdexamfetamine dimesylate and lislevoamfetamine dimesylate, from which the lisdexamfetamine dimesylate selectively crystallized.

In another preferred embodiment, the invention comprises (i) reacting BOC-L-Lys(BOC)—OH with an amine base such as triethylamine (Et₃N) in organic solvent to obtain, e.g. BOC-L-Lys(BOC)—O-Et₃NH+, and then (ii) deprotecting diethyl(1-phenylpropan-2-yl)phosphoramidate (S- or R- or a racemic mix of S-, R-) with aqueous HCl and NaOH to produce an aqueous solution of dextroamphetamine ((2S)-1-phenylpropan-2-amine), levoamphetamine ((2R)-1-phenylpropan-2-amine) or a racemic mixture of aqueous dextroamphetamine and levoamphetamine and then (iii) reacting BOC-L-Lys(BOC)—O-Et₃NH+ with the amphetamine (dextroamphetamine, levoamphetamine or the racemic mixture) in the presence of a coupling agent such as DMTMM ((4-(4,6-dimethoxy-1,3,5-triazin-2-yl)-4-methyl-morpholinium chloride) and heating the biphasic mixture at 40-50° C. to couple the amphetamine (dextroamphetamine or levoamphetamine or the mixture) with the protected lysine carboxylic acid and yield respective the amides BOC-L-Lys(BOC)—N-dextroamphetamine (or BOC-L-Lys(BOC)—N-levoamphetamine or the racemic mixture), and then (iv) reacting the BOC-L-Lys(BOC)—N-dextroamphetamine (or levoamphetamine or the racemic mixture) with methanesulfonic acid (MsOH) in isopropanol, and crystallizing the product to obtain lisdexamfetamine dimesylate, lislevoamfetamine dimesylate or a racemic mixture of lisdexamfetamine dimesylate and lislevoamfetamine dimesylate, from which the lisdexamfetamine dimesylate selectively crystallized.

### DETAILED DESCRIPTION OF THE PREFERRED EMBODIMENTS

In a preferred embodiment, the invention comprises a method of preparing a lysine-amphetamine amide compound mixing an aqueous amphetamine compound and directly coupling with a triazine activated protected lysine compound.

In a preferred embodiment, the invention comprises a process for making a lysine-amphetamine compound, comprising the steps: (i) reacting a protected L-Lysine ammonium salt BOC-L-Lys(BOC)—O—X—NH+ in an organic solvent with an amphetamine in aqueous base in the presence of a coupling agent to form a biphasic mixture and heating the biphasic mixture at 30-50° C., preferably 40-50° C., to yield BOC-L-Lys(BOC)—N-amphetamine and then (ii) reacting under appropriate conditions the BOC-L-Lys(BOC)—N-amphetamine with methanesulfonic acid (MsOH), and crystallizing the product to obtain lisdexamfetamine dimesylate.

In a preferred embodiment, the invention comprises a lysine-amphetamine compound made by the process above and described herein.

In a preferred embodiment, the invention comprises a method of preparing a lysine-amphetamine compound mixing an amphetamine compound of Formula I with a triazine activated protected L-lysine compound of Formula II under conditions to provide pure crystalline L-lysine-amphetamine amide of Formula III directly from the reaction mixture,
where Formula I (Compound 3) is represented by:

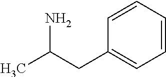

or a salt thereof;
and Formula II is represented by:

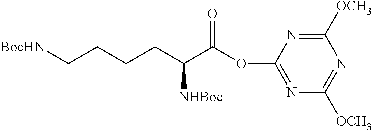 [SMILES: COc1nc(OC)nc(OC(=O)[C@H](CCCCNC(=O)OC(C)(C)C)NC(=O)OC(C)(C)C)n1]

and Formula III is represented by:

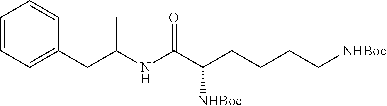 [SMILES: CC(Cc1ccccc1)NC(=O)[C@H](CCCCNC(=O)OC(C)(C)C)NC(=O)OC(C)(C)C]

and then acid deprotected to provide the L-lysine amphetamine amide salt of Formula IV

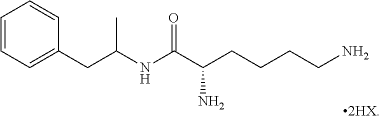 [SMILES: CC(Cc1ccccc1)NC(=O)[C@@H](N)CCCCN]

In another preferred embodiment, the invention is a process as described where Formula I is dextroamphetamine

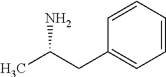

having a purity of at least 90% ee
and Formula III is:

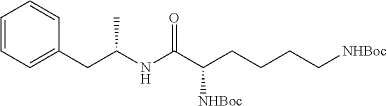 [SMILES: C[C@@H](Cc1ccccc1)NC(=O)[C@H](CCCCNC(=O)OC(C)(C)C)NC(=O)OC(C)(C)C]

and Formula IV is:

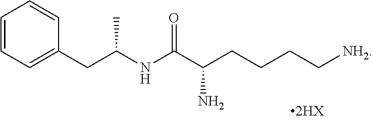 [SMILES: C[C@@H](Cc1ccccc1)NC(=O)[C@@H](N)CCCCN]

In another preferred embodiment, the invention is a process comprising: (i) reacting BOC-L-Lys(BOC)—OH with triethylamine (Et₃N) in methyl tert butyl ether (MTBE) to obtain BOC-L-Lys(BOC)—O-Et₃NH+, and then (ii) deprotecting diethoxy-phosphoramidate with aqueous HCl and NaOH to produce aqueous dexamphetamine ((2S)-1-phenylpropan-2-amine), and then (iii) reacting BOC-L-Lys(BOC)—O-Et₃NH+ with dexamphetamine in the presence of DMTMM ((4-(4,6-dimethoxy-1,3,5-triazin-2-yl)-4-methyl-morpholinium chloride) and heating the biphasic mixture at 40-50° C. to couple the dexamphetamine with the protected lysine carboxylic acid and yield BOC-L-Lys(BOC)—N-dexamphetamine, and then (iv) reacting the BOC-L-Lys(BOC)—N-dexamphetamine with methanesulfonic acid (MsOH) in isopropanol, and crystallizing to obtain lisdexamfetamine dimesylate.

In another preferred embodiment, the invention comprises a process described by the following Scheme I—phosphoramidate source.

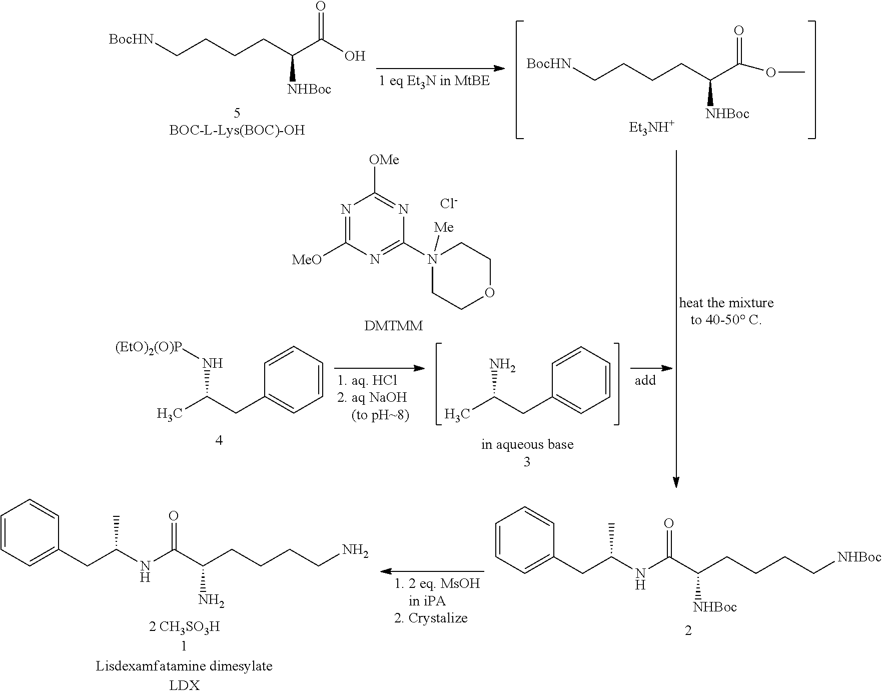

In another preferred embodiment, the invention is a process comprising: (i) reacting BOC-L-Lys(BOC)—OH with triethylamine (Et₃N) in methyl tert butyl ether (MTBE) to obtain BOC-L-Lys(BOC)—O-Et₃NH+ and then (ii) deprotecting a racemic mixture of (S-, R-)-diethoxy-phosphoramidate with aqueous HCl and NaOH to produce an aqueous racemic mixture of dexamphetamine ((2S)-1-phenylpropan-2-amine) and levoamphetamine ((2R)-1-phenylpropan-2-amine), and then (iii) reacting BOC-L-Lys(BOC)—O-Et₃NH+ with the aqueous racemic mixture of dexamphetamine and levoamphetamine in the presence of DMTMM ((4-(4,6-dimethoxy-1,3,5-triazin-2-yl)-4-methyl-morpholinium chloride) and heating the biphasic mixture at 40-50° C. to couple the dexamphetamine and levoamphetamine with the protected lysine carboxylic acid to yield a racemic mixture of BOC-L-Lys(BOC)—N-dexamphetamine and BOC-L-Lys(BOC)—N-levoamphetamine, and then (iv) reacting the racemic mixture of BOC-L-Lys(BOC)—N-dexamphetamine and BOC-L-Lys(BOC)—N-levoamphetamine with MsOH in isopropanol, producing a diastereomeric mixture of lisdexamfetamine dimesylate and lislevoamfetamine dimesylate of which lisdexamfetamine dimesylate can be selectively crystallized. This crystallization occurred without the addition of any single isomer seed crystals, or any other optical resolution agents.

In another preferred embodiment, the invention comprises crystalline lisdexamfetamine dimesylate having a purity ratio of (95:5) or (98.8:1.2) or (99.9: <0.1) of lisdexamfetamine dimesylate to lislevoamfetamine dimesylate.

In another preferred embodiment, the invention comprises a process described by the following Scheme II—racemic.

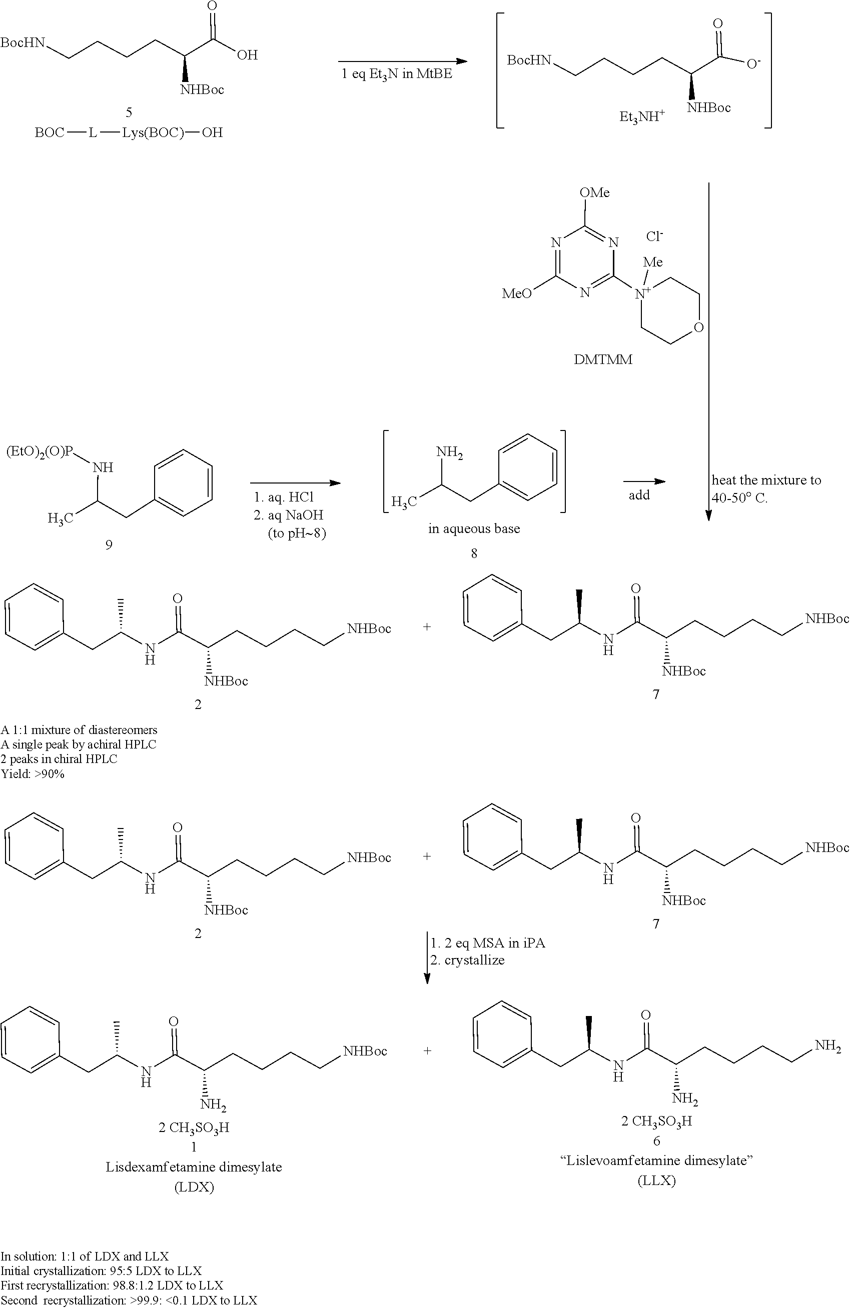

In another preferred embodiment, the invention comprises a process described by the following Scheme III—5 to 1.

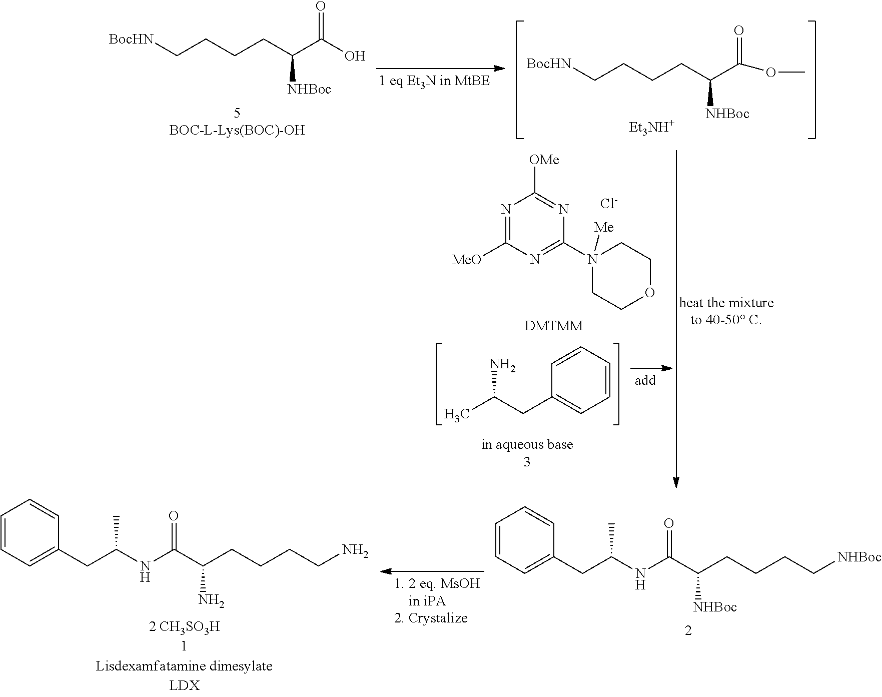

In another preferred embodiment, the invention comprises a process described by the following Scheme IV—5a to 1.

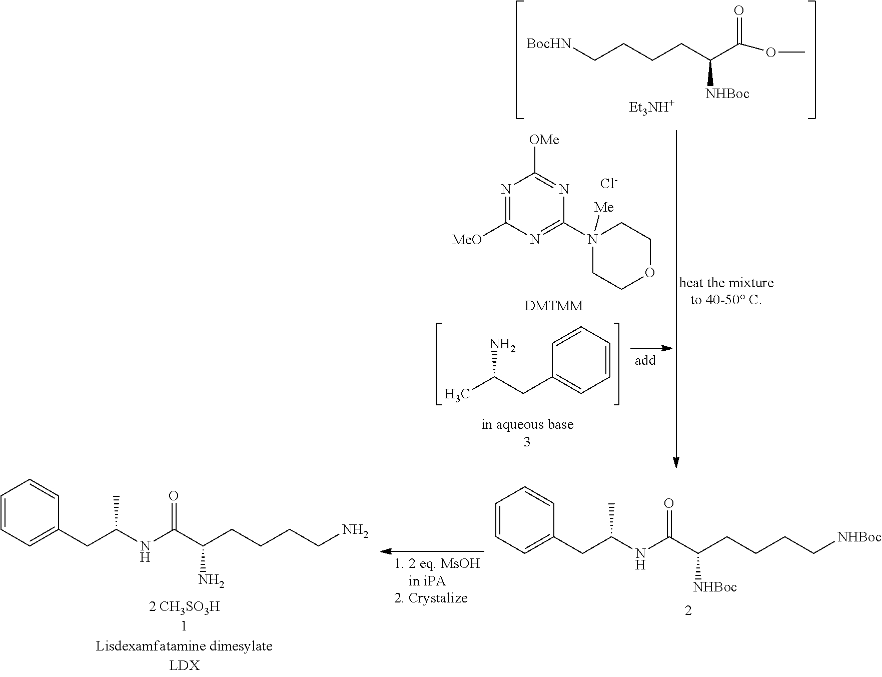

In another preferred embodiment, the invention comprises a process described by the following Scheme V—5a to 2.

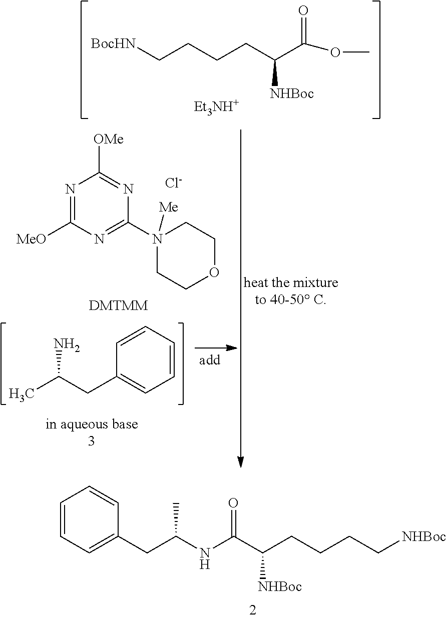

In another preferred embodiment, the invention comprises the in situ generated novel compound, BOC-L-Lys(BOC)-dextroamphetamine as a protected L-lysine amphetamine amide salt.

In another preferred embodiment, the invention comprises the in situ generated novel compound, BOC-L-Lys(BOC)—O-DMT as the preferred amide coupling agent.

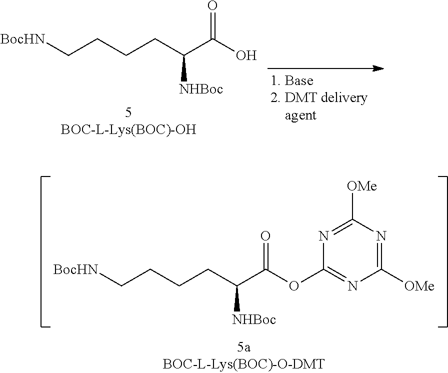

In a preferred embodiment, the invention comprises a mixture of two enantiomers with diastereomers of each comprising levoamphetamine-L-lysine, levoamphetamine-D-lysine, dexamphetamine-L-lysine, and dexamphetamine-D-lysine, and in particular a mixture made by preparing a lysine-amphetamine compound mixing an aqueous amphetamine compound and directly coupling with a triazine activated protected lysine compound.

In another preferred embodiment, the invention comprises a fluidic process, comprising: preparing an aqueous solution of free base amphetamine, coupling the amphetamine with a BOC-L-Lys(BOC)—OH in MTBE and DMTMM in MTBE, heating the aqueous mixture to 50° C. to obtain a biphasic mixture.

In another preferred embodiment, the invention comprises a pharmaceutical composition comprising crystalline lisdexamfetamine dimesylate as described herein and a pharmaceutically acceptable carrier.

In another preferred embodiment, the invention comprises a method of treating attention deficit hyperactivity disorder in a patient in need thereof, comprising the steps of orally administering the pharmaceutical composition described herein.

### Dependent Claims

Any of the processes herein may comprise where the amphetamine is dextroamphetamine, levoamphetamine, or a racemic mixture (d-, l-amphetamine).

Any of the processes herein may comprise where the amphetamine is dextroamphetamine.

Any of the processes herein may comprise where the BOC-L-Lys(BOC)—N-amphetamine is BOC-L-Lys(BOC)—N-dextroamphetamine, BOC-L-Lys(BOC)—N-levoamphetamine, or a racemic mixture (BOC-L-Lys(BOC)—N-(d-,l-)amphetamine.

Any of the processes herein may comprise where the BOC-L-Lys(BOC)—N-amphetamine is BOC-L-Lys(BOC)—N-dextroamphetamine.

Any of the processes herein may comprise where the protected L-Lysine ammonium salt BOC-L-Lys(BOC)—O—X—NH+ is prepared under chemically sufficient conditions using an amine base X selected from the group consisting of triethylamine, N,N-diisopropylethylamine amine, 1,4-diazabicyclo[2.2.2]octane (DABCO) or N-methyl morpholine.

Any of the processes herein may comprise where the protected L-Lysine ammonium salt BOC-L-Lys(BOC)—O—X—NH+ is prepared under chemically sufficient conditions where X is triethylamine to form BOC-L-Lys(BOC)—OEt3NH+.

Any of the processes herein may comprise where the BOC-L-Lys(BOC)—O—X—NH+ is prepared by reacting BOC-L-Lys(BOC)—OH with an amine base where X is triethylamine (Et3N) in methyl tert butyl ether (MTBE) to obtain BOC-L-Lys(BOC)—OEt3NH+.

Any of the processes herein may comprise where the coupling agent is DMTMM ((4-(4,6-dimethoxy-1,3,5-triazin-2-yl)-4-methyl-morpholinium chloride).

Any of the processes herein may comprise where the coupling agent is selected from the group consisting of: 2-chloro-4,6-dimethoxy-1,3,5-triazine (CDMT) or 4-(4,6-Dimethoxy-1,3,5-triazin-2-yl)-4-methylmorpholin-4-ium chloride (DMTMM) or 4-(4,6-Dimethoxy-1,3,5-triazin-2-yl)-4-methylmorpholin-4-ium tosylate (DMTMMOTs), 4-(4,6-Dimethoxy-1,3,5-triazin-2-yl)-4-methylmorpholin-4-ium tetrafluoroborate (DMTMMBF₄), 1-Ethyl-3-(3-dimethylaminopropyl)carbodiimide (EDCI)/1-hydroxybenzotriazole (HOBt), and a mixture thereof.

Any of the processes herein may comprise where the organic solvent is water immiscible and is selected from the group consisting of: Methyl t-butyl ether (MTBE), N,N-dimethylformamide (DMF), dichloromethane (DCM), toluene, acetonitrile, ethyl acetate, isopropanol, isopropyl acetate, cyclohexane, methylene chloride, diethyl ether, tetrahydrofuran (THF), and a mixture thereof.

Any of the processes herein may comprise where the dextroamphetamine is prepared by deprotecting diethoxy-phosphoramidate with aqueous HCl and NaOH to produce aqueous dexamphetamine ((2S)-1-phenylpropan-2-amine).

Any of the processes herein may comprise where the racemic mixture (d-, l-amphetamine) is prepared by deprotecting a racemic mixture of (S-, R-)-diethoxy-phosphoramidate with aqueous HCl and NaOH to produce an aqueous racemic mixture of dexamphetamine ((2S)-1-phenylpropan-2-amine) and levoamphetamine ((2R)-1-phenylpropan-2-amine).

Any of the processes herein may comprise where crystallizing is performed using a crystallization solution comprising isopropanol in isopropyl acetate, and after crystallization yields a 95:5 ratio (purity) of lisdexamfetamine dimesylate to lislevoamfetamine dimesylate.

Any of the processes herein may comprise where crystallizing is performed using a crystallization solution comprising isopropanol in isopropyl acetate, and after a re-crystallization yields a 98.7:1.2 ratio (purity) of lisdexamfetamine dimesylate to lislevoamfetamine dimesylate.

Any of the processes herein may comprise where crystallizing is performed using a crystallization solution comprising isopropanol in isopropyl acetate, and after a second re-crystallization yields a 99.9: <0.1 ratio (purity) of lisdexamfetamine dimesylate to lislevoamfetamine dimesylate.

Any of the processes herein may comprise where the isopropanol is selected from the group consisting of isopropanol (absolute), isopropanol (commercial 91% solution), n-propanol, and a mixture thereof.

Any of the processes herein may comprise where the source of dextroamphetamine is a salt or a phosphoramidate ester.

Any of the processes herein may comprise where the source of the dextroamphetamine salt is selected from the group consisting of: dextroamphetamine hydrochloride, dextroamphetamine sulfate, dextroamphetamine saccharate or dextroamphetamine tartrate.

Any of the processes herein may comprise where the source of the dextroamphetamine salt is dextroamphetamine sulfate.

Any of the processes herein may comprise where the amphetamine phosphoramidate is selected from the group consisting of dimethyl(1-phenylpropan-2-yl)phosphoramidate, diethyl(1-phenylpropan-2-yl)phosphoramidate, diisopropyl(1-phenylpropan-2-yl)phosphoramidate or diphenyl(1-phenylpropan-2-yl)phosphoramidate

Any of the process herein where the amphetamine phosphoramidate can be selected from the group of dextroamphetamine phosphoramidate ester, levoamphetamine phosphoramidate ester or racemic amphetamine phosphoramidate ester.

Any of the process herein where the source of the dextroamphetamine is from the group of: (S)-dimethyl(1-phenylpropan-2-yl)phosphoramidate, (S)-diethyl(1-phenylpropan-2-yl)phosphoramidate, (S)-diisopropyl(1-phenylpropan-2-yl)phosphoramidate or (S)-diphenyl(1-phenylpropan-2-yl)phosphoramidate.

Any of the process herein where the preferred source of the dextroamphetamine is from the group of: (S)-diethyl(1-phenylpropan-2-yl)phosphoramidate.

Any of the processes herein may comprise where a heated organic solution of a carboxylate salt of the bis BOC protected L-Lysine is treated with an aqueous basic solution of dextroamphetamine and this mixture is then treated with an organic solution of a triazine coupling reagent, and this aqueous-organic mixture is heated until the reaction is complete.

Any of the processes herein may comprise where the coupling agent is a triazine coupling reagent and is selected from 2-chloro-4,6-dimethoxy-1,3,5-triazine (CDMT) or 4-(4,6-Dimethoxy-1,3,5-triazin-2-yl)-4-methylmorpholin-4-ium chloride (DMTMM) or 4-(4,6-Dimethoxy-1,3,5-triazin-2-yl)-4-methylmorpholin-4-ium tosylate (DMTMMOTs) or 4-(4,6-Dimethoxy-1,3,5-triazin-2-yl)-4-methylmorpholin-4-ium tetrafluoroborate (DMTMMBF₄).

Any of the process herein may comprise wherein the coupling agent is 1-Ethyl-3-(3-dimethylaminopropyl)carbodiimide (EDCI)/1-hydroxybenzotriazole (HOBt).

Any of the processes herein may comprise where the coupling agent is DMTMM.

Any of the processes herein may comprise where DMTMM where it is prepared in the reaction of CDMT and n-methyl morpholine.

Any of the processes herein may comprise where the carboxylate salt of the BOC-L-Lys(BOC)—OH is selected from the group consisting of sodium, potassium or triethylammonium salts.

Any of the processes herein may comprise where the carboxylate salt of the BOC-L-Lys(BOC)—OH is the triethylammonium salt.

Any of the processes herein may comprise where organic solvent is either water miscible or water immiscible.

Any of the processes herein may comprise where organic solvent is water immiscible and is selected from the group consisting of Methyl t-butyl ether (MTBE), N,N-dimethylformamide (DMF), dichloromethane (DCM), toluene, acetonitrile, ethyl acetate, isopropanol, isopropyl acetate, cyclohexane, methylene chloride, diethyl ether, tetrahydrofuran (THF), and a mixture thereof.

Any of the processes herein may comprise where the solvent is selected from the group consisting of toluene, methylene chloride, MTBE, ethyl acetate or isopropyl acetate.

Any of the processes herein may comprise where the organic component is a mixture of the BOC protected lysine and a base.

Any of the processes herein may comprise where the base is selected from the group consisting of a sodium base, potassium base, lithium base or an amine base.

Any of the processes herein may comprise where the sodium base is selected from the group consisting of sodium bicarbonate, sodium carbonate, sodium hydride, sodium amide, sodium methoxide or sodium hydroxide and the preferred base is sodium bicarbonate.

Any of the processes herein may comprise where the potassium base is selected from the group consisting of potassium bicarbonate, potassium carbonate, potassium t-butoxide or potassium hydroxide and the preferred base is potassium bicarbonate.

Any of the processes herein may comprise where the amine base is selected from the group consisting of triethylamine, N,N-diisopropylethylamine amine, 1,4-diazabicyclo[2.2.2]octane (DABCO) or N-methyl morpholine. The preferred organic base is triethylamine.

Any of the processes herein may comprise where organic solvent is water miscible and is selected from the group consisting of methanol, ethanol, isopropanol, n-propanol, acetonitrile, THF, N,N-dimethylformamide (DMF) or dimethyl sulfoxide.

Any of the processes herein may comprise where the water miscible solvent is selected from the group consisting of ethanol, isopropanol, n-propanol, THF or acetonitrile.

Any of the processes herein may comprise where the water miscible solvent of choice is isopropanol.

Any of the processes herein may comprise where the organic component is a mixture of the BOC protected lysine and a base.

Any of the processes herein may comprise where the base is selected from the group consisting of a sodium base, potassium base, lithium base or an amine base.

Any of the processes herein may comprise where the sodium base is selected from the group consisting of sodium bicarbonate, sodium carbonate, sodium methoxide or sodium hydroxide and the preferred base is sodium bicarbonate.

Any of the processes herein may comprise where the potassium base is selected from the group consisting of potassium bicarbonate, potassium carbonate, potassium t-butoxide or potassium hydroxide and the preferred base is potassium bicarbonate.

Any of the processes herein may comprise where the amine base is selected from the group consisting of triethylamine (Et₃N), N,N-diisopropylethylamine in ethanol, 1,4-diazabicyclo[2.2.2]octane (DABCO) or N-methyl morpholine. The preferred organic base is triethylamine.

Any of the processes herein may comprise where the L-lysine amphetamine amide salt of Formula IV is crystallized using isopropanol (both anhydrous or a commercial 91% isopropyl alcohol solution), n-propanol, or N,N-dimethylformamide.

Any of the processes herein may comprise where L-lysine amphetamine amide salt of Formula IV is crystallized using isopropanol (both anhydrous or a commercial 91% isopropyl alcohol solution), n-propanol, or N,N-dimethylformamide, and after crystallization yields a 95:5 ratio (purity) of lisdexamfetamine dimesylate to lislevoamfetamine dimesylate.

Any of the processes herein may comprise where L-lysine amphetamine amide salt of Formula IV is re-crystallized and yields a 98.8:1.2 ratio (purity) of lisdexamfetamine dimesylate to lislevoamfetamine dimesylate.

Any of the processes herein may comprise where L-lysine amphetamine amide salt of Formula IV is re-crystallized a second time and yields a 99.9: <0.1 ratio (purity) of lisdexamfetamine dimesylate to lislevoamfetamine dimesylate.

Any of the processes herein may comprise where the process yields 85-90%,

Any of the processes herein may comprise where the process includes at least one re-crystallization step.

Any of the processes herein may comprise where the racemic process after crystallization yields a 95:5 ratio (purity) of lisdexamfetamine dimesylate to lislevoamfetamine dimesylate.

Any of the processes herein may comprise where the process after a first re-crystallization yields a 98.8:1.2 ratio (purity) of lisdexamfetamine dimesylate to lislevoamfetamine dimesylate.

Any of the processes herein may comprise where the process after a second re-crystallization yields a 99.9: <0.1 ratio (purity) of lisdexamfetamine dimesylate to lislevoamfetamine dimesylate.

### HPLC Methods

### Achiral HPLC Method:

Column: YMC ODS-AQ S5 120 A (250×4.6 mm×5 μm, YMC, Nagoya, Japan)
Detector: 215 nm
Mobile phase: Mobile phase A: 0.1% methanesulfonic acid (v/v) in water; Mobile phase B: 0.1% methanesulfonic acid in acetonitrile. The HPLC gradient program was set as follows: Time (min)/% of solvent B: 0/3, 15/20, 30/50, 35/95, 37/95, 37.1/3, 45/3. The flow rate was 1.0 mL/min for a total run time of 45 minutes.

Flow rate 1 mL/min.

Sample preparation: 1.0 mg/mL of substrate in a water and acetonitrile mixture (70:30, v/v).

### Chiral HPLC Method:

Column: Chiralpak IA. P/N 80325, 4.6 mm i.d.×250 nm, 5 μm particle size (Daicel Chiral
Technologies)
Column temperature: Ambient
Detection: 215 nm
Mobile phase: 96% heptane: 4% 3 A anhydrous ethanol (3% methanol denatured), Isocratic
Flow rate 1 mL/min
Analysis time: 30 min
Sample preparation: accurately weigh 100 mg of sample into a 100 mL of volumetric flask and dissolve sample in 10 mL of 3 A anhydrous ethanol and diluted with 90 mL of heptane.

### Example—Preparation of Lisdexamfetamine Dimesylate (1) from Bis-BOC-Lisdexamfetamine (2)

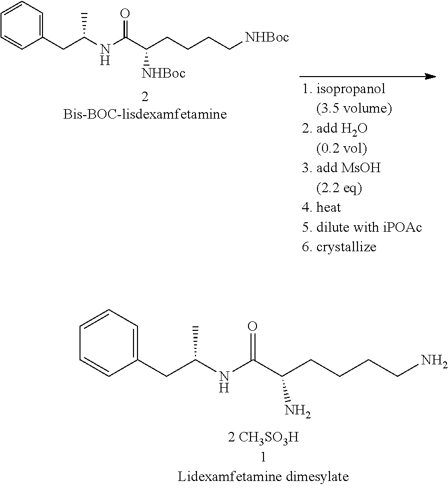 [SMILES: C[C@@H](Cc1ccccc1)NC(=O)[C@H](CCCCNC(=O)OC(C)(C)C)NC(=O)OC(C)(C)C ; C[C@@H](Cc1ccccc1)NC(=O)[C@@H](N)CCCCN]

Powdered 2 (50 g, 107 mmol) was dissolved in IPA (175 mL) in a 1 L 3 necked round bottomed flask with overhead stirring and a reflux condenser. The stirrer was started and to this solution was charged water (12.5 mL; 0.25 volumes). Methanesulfonic acid (15.5 mL, 240.0 mmol) with a syringe pump. The solution was heated to 80° C. The reaction was monitored by HPLC and after complete conversion, isopropyl acetate (175 mL) was added to induce crystallization. The resulting slurry was allowed to cool to room temperature and stirred for 1 hour.

The solid 1 was isolated by filtration and the reaction flask and filter cake was washed with a 30% isopropanol in isopropyl acetate (200 mL). The filter cake was dried in vacuo (at 30° C.) for 2 hours affording lisdexamfetamine dimesylate (1) as a white solid (45.6 g, 92.8% yield)

### Example—Preparation of 2 from Dextroamphetamine Sulfate

### Reaction Solution A (Compound 3 in Aqueous Basic Solution):

A 500 mL 3-necked round bottomed flask equipped with a water-cooled condenser, magnetic stir bar and thermometer was charged with 20.0 g (0.11 mol) of dextroamphetamine sulfate. To this solid was added saturated sodium bicarbonate solution (150 mL). Stirring was initiated and maintained for 1 hr. The suspension slowly turned into a homogeneous, clear solution. This was Reaction Solution A.

### Reaction Solution B (5 Triethylammonium Salt in MTBE):

In a separate vessel, BOC-L-Lys(BOC)—OH (5) (45.7 g, 1.17 eq) was added MTBE (400 mL) and stirred until the acid dissolved. To this solution was added triethylamine (15.9 g, 1.4 eq). Stirring was continued for 30 minutes affording a clear, colorless solution. This solution of 5 triethylammonium salt in MTBE was ready to be used in subsequent conversion as Reaction Solution B.

### The Condensation Reaction:

To Reaction Solution B was added solid DMTMM (4-(4,6-Dimethoxy-1,3,5-triazin-2-yl)-4-methylmorpholinium chloride) (39.1 g, 1.2 eq after correction for residual water). The stirring was continued, and the MTBE solution was heated to 35° C. affording a clear solution. To this heated solution was added Reaction Solution A and the temperature was raised to 45-50° C. and stirred for three hours. HPLC analysis (achiral HPLC method) showed over 98% conversion to 2. The 45° C. batch was diluted with water (50 mL) was added and the reaction was transferred to a separatory funnel and the layers were separated. The MTBE layer was washed with saturated sodium chloride solution (100 mL), dried over sodium sulfate, filtered and evaporated to dryness affording a white solid. This solid was dissolved in dichloromethane (150 mL) and stirred for 1 hr. To this solution was added heptane (300 mL) and the resulting slurry was cooled to 5-10° C. and aged for 1 hr. The solids were collected by filtration, dried under reduced pressure to afford 2 (44.8 g, 88.0% yield) as a white solid. The NMR spectrum matched the literature spectrum. The purity of 2 was 96.8% by achiral HPLC analysis and had an optical purity of 100% by chiral HPLC analysis.

### Comparative Example—Prior Art

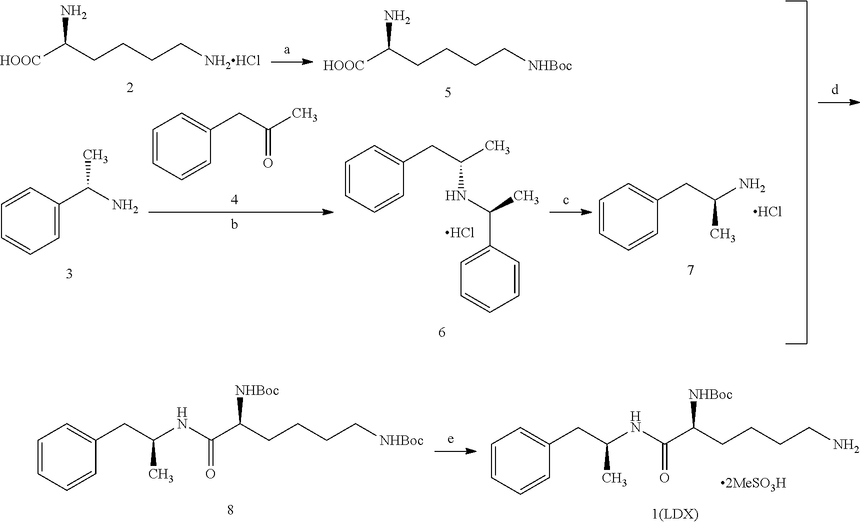

The synthesis route of LDX. Reagents and conditions: (a) Boc₂O, acetone/2N NaOH, 25° C., 4 h, 94%-98%; (b) (i) NaBH(AcO) 3, DCM, rt, 9 h; (ii) THF, 36% hydrochloric acid, 73%--78%; (c) ammonium formate, MeOH, 65° C., 3 h, 92%-96%; (d) (i) EDCL HOBt, NMM, DMF rt., 20 h, 92-95%; (ii) recrystallization (acetone:n-heptane 1:10, v/v); (e) MeSO₃H, THF, 50° C., 6 h, 95%-96%.

### Example—Preparation of LDX (1) from BOC-L-Lys(BOC)—OH (5) and Deprotected diethoxy-phosphoramidate (4->3)

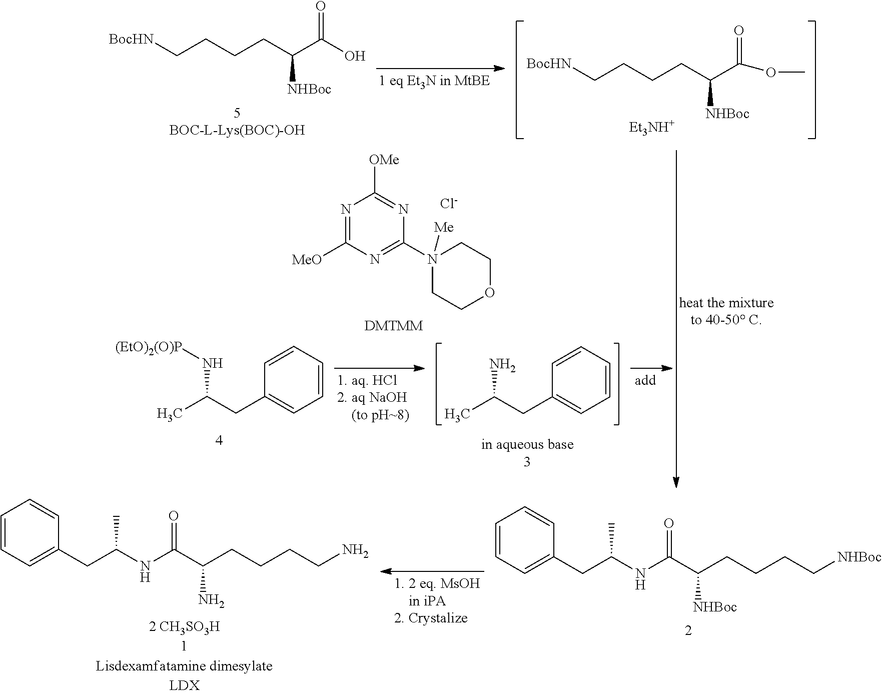

### Example—Process for BOC-L-Lys(BOC)—OH (5)—Isolated Yield: 92%

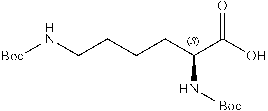

A 2 L three-neck flask equipped with a mechanical stirrer was charged with a solution of L-lysine monohydrochloride (50.03 g, 274 mmol) in water (275 ml), 1,4-dioxane (275 ml) and 1 M sodium hydroxide solution (275 mmol, 275 ml) (resulting: pH 10.0). To this solution was added di-tert-butyl dicarbonate (179 g, 820 mmol) in one portion whilst stirring. The evolution of gas was noted, and the pH of the reaction mixture slowly decreased. A Metrohm Titrino 702 SM dosing unit was set up in “Set Endpoint Titration” mode in order to keep the pH of the reaction mixture constant at pH 7.30 by automatically dosing 1 M NaOH. Note: the reaction mixture apparently formed a buffered system at pH 7.05.

LCMS analysis (NQAD detection) indicated near-complete consumption of the lysine starting material after a reaction time of 20 h. The reaction mixture was concentrated by rotary evaporation to a total volume of about 500 mL. Water (250 mL) was added (resulting pH: 8.8) and then EtOAc was added (250 mL). Solid KHSO₄ (about 70 g) was added in portions until a pH of 2.3 was reached (Note: evolution of gas; the mixture formed a buffer at pH 2.3). The phases were separated, the aqueous layer was extracted with EtOAc (2×250 mL), and the combined organic phases were washed sequentially with 0.25 M KHSO₄ solution (2×100 mL), water (50 mL) and saturated aqueous sodium chloride solution (2×50 mL). Drying over Na₂SO₄ and concentration in vacuo yielded the title compound as a clear, colorless, sticky mass (98.18 g).

LCMS: Purity (NQAD detection): 99%, mass in agreement with molecular formula (neg. m/z=345 [M−1]−, 691 [2M−1]−).

TLC (EtOAc/heptane=1/1 containing 2 vol % AcOH, anisaldehyde staining): One major spot (Rf 0.28), with no di-tert-butyl dicarbonate present. A small impurity with a higher Rf was visible.

Kaiser test: negative (no primary amines present).

### Example—Process for 5 from L-Lysine HCl (Dioxane Free Process)

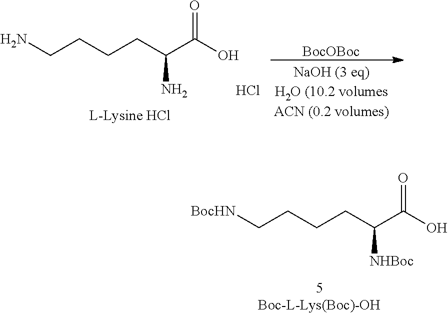 [SMILES: "BocOBoc" ; NCCCC[C@H](N)C(=O)O ; Cl ; CC(C)(C)OC(=O)NCCCC[C@H](NC(=O)OC(C)(C)C)C(=O)O]

To a water cooled, stirred solution of L-lysine monohydrochloride (100 g,) in water (1 L) and acetonitrile (100 mL) was added solid sodium hydroxide (66 g, 3 eq) followed by di-tert-butyl dicarbonate (280 g, 2.3 eq). The temperature of the stirred solution was raised to 55° C. and held overnight. The reaction mixture was cooled to room temperature and washed with toluene (2×100 mL), and these toluene washes were discarded. To the remaining aqueous solution was added MTBE (500 mL) and stirred biphasic mixture cooled to 10° C. and the pH of the solution was adjusted 6-7 by slowly adding 4 N HCl (200 mL). When the foaming ceased, the layers were separated, and the MTBE was discarded. The aqueous layer was returned to the flask and fresh MTBE (200 mL) was added. The stirred biphasic mixture was cooled to 15-20° C. and the pH was then adjusted to 2-3 by the slow addition of 4 N HCl (200 mL). The biphasic solution was stirred for 1 hour and the layers were allowed to settle for 1 hour and separated. The separated aqueous layer was back extracted with MTBE (100 mL).

The combined MTBE organic layers were washed with water (2×100 mL) and the water washes were discarded. The remaining organic phase was dried (MgSO4), filtered and concentrated under reduced pressure to afford 5 (175 g, 93% yield but it still contains some MTBE by proton NMR). The product was diluted in MTBE to afford Reaction Solution A.

### Example—Preparation of 4-(4,6-dimethoxy-1,3,5-triazin-2-yl)-4-methylmorpholinium chloride (DMTMM)

A suspension of 2-chloro-4,6-dimethoxy-1,3,5-triazine (250 g) in MTBE (1.75 L) was cooled to about 10° C. To this suspension was slowly added a solution of N-methylmorpholine (183.4 g) in MTBE (750 mL) while maintaining the batch temperature below 10° C. The resulting thicker slurry was stirred for 15-30 minutes. The solid product was collected by filtration and the flask and filter cake was washed with cold MTBE (100 mL). The solid was dried in a room temperature vacuum oven affording crude DMTMM (378 g, 96% yield) as a light-yellow solid which was 99.2% pure by achiral HPLC. The NMR was consistent with the literature NMR spectrum. This material was acceptable to use in the subsequent condensation reaction.

If recrystallization is needed: The DMTMM (100 g of a 78% pure DMTMM batch) was dissolved in methanol (200 mL). The solution was filtered and to the filtrate was added MTBE (300 mL) and cooled to about 10° C. and the solid product was collected by filtration. The slurry flask and filter cake were washed with a chilled 10% methanol in MTBE solution (100 mL). The solid was dried under reduced pressure to afford purified DMTMM (75 g, 75% recovery) of purified DMTMM. The HPLC analysis was a single peak for DMTMM. The analytical data was consistent with the literature.

### Example—Preparation of 4-(4,6-dimethoxy-1,3,5-triazin-2-yl)-4-methylmorpholinium tosylate (DMTMMOTs)

To a solution of 4-methylbenzene sulfonic acid (3.80 g, 20 mmol) and sodium bicarbonate (5.04 g, 60 mmol) in acetonitrile (60 mL) was added 2-chloro-4,6-dimethoxy-1,3,5-triazine (3.50 g, 20 mmol) and stirred for 10 minutes. N-methylmorpholine (2.02 g, 2.2 mL, 20 mmol) was added and the slurry was stirred at 5° C. overnight. The batch was clarified, and the solution was concentrated to dryness at a temperature not exceeding 20° C. The solid residue was recrystallized from a mixture of acetonitrile and diethyl ether to afford a 73% yield of DMTMMOTs (6.0 g) as a white crystalline solid.

### Example—Preparation of 2

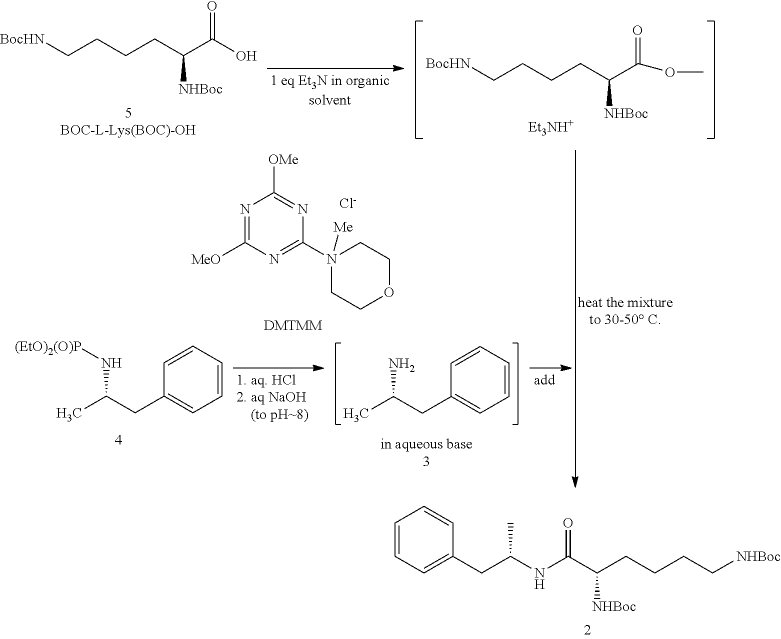

### Example—Preparation of [(S)-5-tert-Butoxycarbonylamino-5-((S)-1-methyl-2-phenyl-ethylcarbamoyl)-pentyl]-carbamic acid tert-butyl ester (2)

### Reaction Solution A:

A 1 L round bottomed flask equipped with a jacketed water condenser, mechanical stirrer and temperature probe was charged with 50.0 g of 4. To this solid was added 3N Hydrochloric acid (220 mL). Stirring was initiated and the resultant suspension was heated to 80° C. and maintained for 2 hr. The suspension slowly turned into a homogeneous, clear solution. The acidic solution was cooled to room temperature and washed with toluene (4×200 mL). The toluene layers were discarded. The aqueous layer was cooled to 10° C.), whereupon a 50% aqueous solution of sodium hydroxide (35.0 g, 4.7 eq) was slowly added. The pH of the solution was adjusted between 7 to 9 and this solution was ready to be used in subsequent conversion as Reaction Solution A.

### Reaction Solution B:

In a separate vessel, 5 (75.0 g, 1.17 eq) was added to MTBE (250 mL) and to this mixture was added triethylamine (26.1 g, 1.4 eq). Stirring was initiated and maintained for 30 minutes affording a clear, colorless solution. This solution was ready to be used in subsequent conversion as Reaction Solution B.

### The Condensation Reaction:

To a 2 L, 3-necked round bottomed flask equipped with a mechanical stirrer, water cooled condenser, temperature probe and addition funnel was charged DMTMM (4-(4,6-Dimethoxy-1,3,5-triazin-2-yl)-4-methylmorpholinium chloride) (66.4 g, 1.1 eq after correction for residual water) followed by MTBE (500 mL). The stirrer was started, and the suspension was heated to 35° C. affording a clear solution. To this solution was added Reaction Solution B and the mixture was stirred for about 30 minutes. To this heated solution was added Reaction Solution A over 15 minutes and the agitation was increased to efficiently mix the biphasic mixture. The temperature was raised to 45-50° C. and stirred for two hours and the reaction was monitored by HPLC. When complete by HPLC analysis, the stirred mixture was diluted with water (250 mL) and MTBE (250 mL) to dissolve any solids that separated. The reaction mixture was heated to 50° C. affording a clear, biphasic mixture. The stirrer was stopped, and the aqueous layer was removed. The organic layer (while still at 50° C.) was washed with warm water (500 mL). The aqueous layer was removed and the organic layer (still at 50° C.) was washed with a 1% aqueous solution of citric acid solution (500 mL). The aqueous layer was separated, and the organic layer (still at 50° C.) was washed with a 2% aqueous sodium bicarbonate solution (500 mL) followed by water washes (2×250 mL) and the aqueous washes were discarded.

The remaining organic layer was concentrated under reduced pressure to about one-third of the original volume (removed −350 mL of MTBE) The remaining slurry was cooled to room temperature and heptanes (300 mL) was added and mechanically stirred for 5 h. The batch was cooled to about 5° C. for 30 min and subsequently filtered. The collected solid was washed with a mixture of 30% MTBE in heptane (7:3, 150 mL total) and dried under vacuum affording 79.0 g (92% yield) of the desired 2 as a white, crystalline solid. The NMR spectrum matched the literature spectrum. The purity of 2 was 99.57% by achiral HPLC analysis and had an optical purity of 100% by chiral HPLC analysis.

### Example—Preparation of 1 from 2

To a 5 L, 4-neck round bottomed flask equipped with a mechanical stirrer, temperature probe and a jacketed water condenser was charged with 2 (465.2 g) followed by isopropanol (1.64 L) and water (125 mL). Stirring was initiated and to the reaction mixture was added methanesulfonic acid (143.4 mL, 2.2 eq). The resultant solution was heated to 80° C. and therein maintained for 12 hrs. The reaction mixture was treated with isopropyl acetate (3.2 L) which was added over 1 hour while maintaining the batch temperature. The mixture was then cooled to 55° C., over 1 hr. and stirred for an additional 2 hr. The mixture was then cooled to 25° C. over the next hour and stirred for an additional 10 hour. The mixture was filtered, washed with a chilled (5-10° C.) mixture of 30% isopropyl alcohol in isopropyl acetate (400 mL) and dried under vacuum affording 419 g (91.7% yield) of 1 as a white, crystalline solid. This solid was 99.8% pure by achiral HPLC analysis and 100% by chiral HPLC analysis (no impurity >0.1%).

If necessary, 1 can be recrystallized:

To a 1 L, 3 necked round bottomed flask with a mechanical stirrer, water cooled reflux condenser and temperature probe was charged 1 (10.g), isopropanol (200 mL) and water (25 mL). The mixture was heated to 80° C. at which point all of the solids dissolved. To this solution was added isopropyl acetate (400 mL) and the slurry was cooled to 25° C. and held for 10 hours. The solid was isolated by filtration and the flask and filter cake washed with chilled isopropyl acetate and dried under vacuum to afford 9.6 g of recrystallized 1 (96% recovery) of 1 as a white solid. The HPLC purity of this sample was 99.96%. The chiral analysis was unchanged.

### Example—Preparation of 2 Using DMTMM OTs

A suspension of 4 (3 g, 11.05 mmol) in 3N HCl (13 mL) was heated to 80° C. at which point a solution was obtained. The heating was continued for about 2 hours until the reaction was complete by HPLC analysis. The solution was cooled to 25° C. and washed with toluene (25 mL). The toluene wash was separated and disposed. The aqueous solution was cooled 10° C. and made basic by the slow addition of 50% NaOH solution to a pH about 9. This is Reaction Solution A.

To a separate flask was charged 5 (4.5 g) and MTBE (15 mL) and triethyl amine (1.56 g; 2.2 mL) was added and stirred for 30 minutes until a clear solution was obtained. To this solution was added a suspension of DMTMM OTs (5.0 g) in MTBE (15 mL). To this solution was added Reaction Solution A at 30° C. and then stirred overnight at 45° C., overnight.

The biphasic mixture was transferred to a separatory funnel and washed with water (2×50 mL) and the MTBE layer was concentrated under reduced pressure and heptane (30 mL) was added. The resulting slurry was cooled to 10° C. and stirred for 2 hours. The solid was collected by filtration and the flask and filter cake was washed with chilled (0-5° C.) 30% MTBE in heptane. The solid was dried under reduced pressure at 30° C. to afford 2 (4.35 g, 85% yield). The NMR spectrum matched the literature spectrum. The purity of 2 was 99.1% by achiral HPLC analysis and had an optical purity of 100% by chiral HPLC analysis.

### Example—Preparation of 2 in Aqueous and Organic Solvent Mixtures

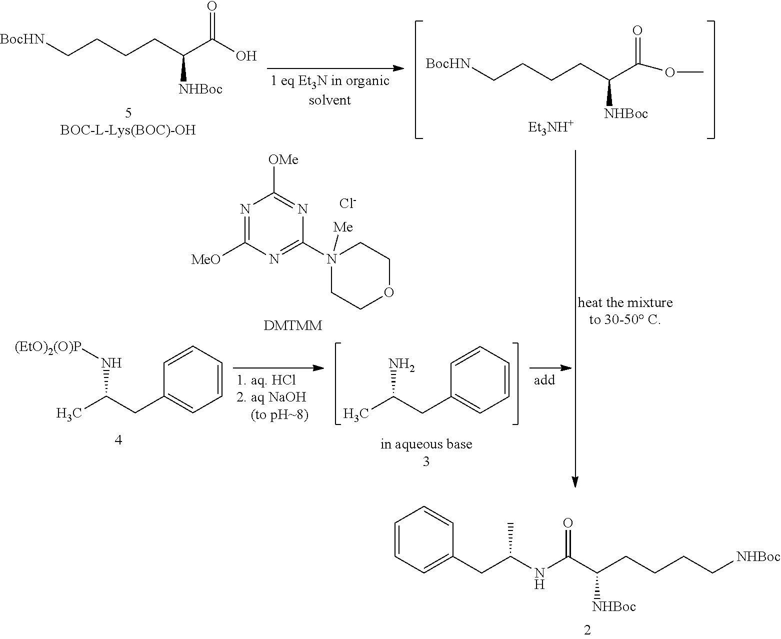

### Example—Preparation of 2 in Ethanol

### Reaction Solution A (Compound 3 in Aqueous Basic Solution):

A 500 mL 3-necked round bottomed flask equipped with a water-cooled condenser, magnetic stir bar and thermometer was charged with 4 (30.0 g). To this solid was added 3N Hydrochloric acid (140 mL). Stirring was initiated and the resultant suspension was heated to 80° C. and maintained for 1 hr. The suspension slowly turned into a homogeneous, clear solution. The acidic solution was cooled to room temperature and washed with toluene (100 mL). The toluene layer was discarded. The aqueous layer was cooled to 10° C., whereupon a 50% aqueous sodium hydroxide solution (250.0 g, 3.4 eq) was slowly added. The pH of the 3 in the aqueous solution was adjusted between 7 to 9 and this solution was ready to be used in subsequent conversion as Reaction Solution A.

### Reaction Solution B (5 Triethylammonium Salt in Ethanol):

In a separate vessel, BOC-L-Lys(BOC)—OH (5) (45.0 g, 1.17 eq) was added to ethanol (300 mL) and stirred until the acid dissolved. To this solution was added triethylamine (15.6 g, 1.4 eq). Stirring was continued for 30 minutes affording a clear, colorless solution. This solution of 5 triethylammonium salt in ethanol was ready to be used in subsequent conversion as Reaction Solution B.

### The Condensation Reaction:

To Reaction Solution B was added solid DMTMM (4-(4,6-Dimethoxy-1,3,5-triazin-2-yl)-4-methylmorpholinium chloride) (42.3 g, 1.2 eq after correction for residual water). The stirring was continued, and the ethanol solution was heated to 35° C. affording a clear solution. To this heated solution was added Reaction Solution A and the temperature was raised to 45-50° C. and stirred for three hours. After 1.5 hours, the 2 began to crystalize. HPLC analysis (achiral HPLC method) showed over 98% conversion to 2. The batch was cooled to 25-30° C. and water (50 mL) was added and the suspension was stirred for one hour and filtered. The filter cake (containing crude 2) was air dried affording 54.0 g of crude 2 a white solid. The air-dried crude 2 was slurried in water (150 mL) for 2 hours and the solids were collected by filtration. The filter cake was dried under reduced pressure at 40° C. affording 2 as a white solid (48.2 g, 92.8% yield). The NMR spectrum matched the literature spectrum. The purity of 2 was 94.5% by achiral HPLC analysis and had an optical purity of 100% by chiral HPLC analysis.

### Example—Preparation of 2 in Isopropanol

### Reaction Solution A (Compound 3 in Aqueous Basic Solution):

A 2 L mL 3-necked round bottomed flask equipped with a water-cooled condenser, magnetic stir bar and thermometer was charged with 4 (100.0 g). To this solid was added 3N Hydrochloric acid (460 mL). Stirring was initiated and the resultant suspension was heated to 80° C. and maintained for 1 hr. The suspension slowly turned into a homogeneous, clear solution. The acidic solution was cooled to room temperature and washed with toluene (250 mL). The toluene layer was discarded. The aqueous layer was cooled to 10° C., whereupon a 50% aqueous sodium hydroxide solution (120 g, 4.7 eq) was slowly added. The pH of the 3 in solution was adjusted between 7 to 9 and this solution was ready to be used in subsequent conversion.

### Reaction Solution B (5 Triethylammonium Salt in Isopropanol):

In a separate 2 L flask, BOC-L-Lys(BOC)—OH (5) (148.5 g, 1.17 eq) was added to isopropanol (900 mL) and stirred until the acid dissolved. To this solution was added triethylamine (43.8 g, 1.4 eq). affording a clear, colorless solution. This solution of 5 triethylammonium salt in isopropanol was ready to be used in subsequent conversion

### The Condensation Reaction:

To stirred Reaction Solution B in a 5 L flask equipped with an overhead stirrer, and reflux condenser was added DMTMM (4-(4,6-Dimethoxy-1,3,5-triazin-2-yl)-4-methylmorpholinium chloride) (139.5 g, 1.07 eq after correction for residual water). The stirred solution heated was heated to 35° C. affording a clear solution. To this heated solution was added Reaction Solution A and the temperature was raised to 45-50° C. After 1.5 hours the reaction product began to crystallize. After 3 hours, HPLC analysis (achiral HPLC method) showed over 98% conversion to 2. The heat was removed, and the reaction was cooled to 20-25° C. and stirred for 2 hours. The solid was collected by filtration and washed with water (2×200 mL). The solid 2 was dried at 40° C. under reduced pressure to afford the 2 as a white solid (144.6 g, 84% yield). The NMR spectrum matched the literature spectrum. The purity of 2 was 98.8% by achiral HPLC analysis and had an optical purity of 100% by chiral HPLC analysis.

### Example—Preparation of 2 in Ethyl Acetate

### Reaction Solution A (Compound 3 in Aqueous Basic Solution):

A 50 mL 3-necked round bottomed flask equipped with a water-cooled condenser, magnetic stir bar and thermometer was charged with 3.0 g of 4. To this solid was added 3N Hydrochloric acid (14 mL). Stirring was initiated and the resultant suspension was heated to 80° C. and maintained for 1 hr. The suspension slowly turned into a homogeneous, clear solution. The acidic solution was cooled to room temperature and washed with toluene (10 mL). The toluene layer was discarded. The aqueous layer was cooled to 10° C., whereupon a 50% aqueous sodium hydroxide solution (35.0 g, 4.7 eq) was slowly added. The pH of the 3 in solution was adjusted between 7 to 9 and this solution was ready to be used in subsequent conversion as Reaction Solution A.

### Reaction Solution B (5 Triethylammonium Salt in Ethyl Acetate):

In a separate vessel, 5 (4.5 g, 1.17 eq) was added to ethyl acetate (20 mL) and stirred until the acid dissolved. To this solution was added triethylamine (1.56 g, 1.4 eq). Stirring was continued for 30 minutes affording a clear, colorless solution. This solution of 5 triethylammonium salt in ethyl acetate was ready to be used in subsequent conversion as Reaction Solution B.

### The Condensation Reaction:

To Reaction Solution B was added DMTMM (4-(4,6-Dimethoxy-1,3,5-triazin-2-yl)-4-methylmorpholinium chloride) (3.96 g, 1.07 eq after correction for residual water). The stirring was continued, and the ethyl acetate solution was heated to 35° C. affording a clear solution. To this heated solution was added Reaction Solution A and the temperature was raised to 45-50° C. and stirred for three hours. HPLC analysis (achiral HPLC method) showed the reaction to be complete. The reaction was diluted with additional ethyl acetate (15 mL).

The biphasic mixture was separated. The organic layer was washed with water (2×2 ml) and the remaining ethyl acetate layer was dried over sodium sulfate. The mixture was clarified, and the ethyl acetates solution was concentrated under reduced pressure to afford a slurry of 2 in ethyl acetate. The slurry was diluted with heptane (40 mL) and the slurry was cooled to 5° C. The solid 2 was collected by filtration and washed with 10% ethyl acetate in heptane (20 mL). The solid was dried under reduced pressure affording 2 (4.65 g, 90% yield). The NMR spectrum matched the literature spectrum. The purity of 2 was 99.3% by achiral HPLC analysis and had an optical purity of 100% by chiral HPLC analysis.

### Example—Preparation of 2 in Acetonitrile

### Reaction Solution A (Compound 3 in Aqueous Basic Solution):

A 50 mL 3-necked round bottomed flask equipped with a water-cooled condenser, magnetic stir bar and thermometer was charged with 3.0 g of 4. To this solid was added 3N Hydrochloric acid (14 mL). Stirring was initiated and the resultant suspension was heated to 80° C. and maintained for 1 hr. The suspension slowly turned into a homogeneous, clear solution. The acidic solution was cooled to room temperature and washed with toluene (10 mL). The toluene layer was discarded. The aqueous layer was cooled to 10° C.), whereupon a 50% aqueous sodium hydroxide solution (35.0 g, 4.7 eq) was slowly added. The pH of the 3 in solution was adjusted between 7 to 9 and this solution was ready to be used in subsequent conversion as Reaction Solution A.

### Reaction Solution B (5 Triethylammonium Salt in Acetonitrile:

In a separate vessel, 5 (4.5 g, 1.17 eq) was dissolved in acetonitrile (20 mL) and stirred until the acid dissolved. To this solution was added triethylamine (1.56 g, 1.4 eq). Stirring was continued for 15 minutes affording a clear, colorless solution. This solution of 5 triethylammonium salt in acetonitrile was ready to be used in subsequent conversion as Reaction Solution B.

### The Condensation Reaction:

To Reaction Solution B was added DMTMM (4-(4,6-Dimethoxy-1,3,5-triazin-2-yl)-4-methylmorpholinium chloride) (3.96 g, 1.07 eq after correction for residual water). The stirring was continued, and the acetonitrile solution was heated to 35° C. affording a clear solution. To this heated solution was added Reaction Solution A and the temperature was raised to 45-50° C. and stirred for two hours. HPLC analysis (achiral HPLC method) showed the reaction to be complete. The reaction was diluted with water (50 mL) and stirred for 30 minutes, filtered and the filter cake was air dried, affording crude 2 (6.8 g) as a light yellow solid. The crude 2 was recrystallized from the MTBE/heptane method affording purified 2 (4.14 g, 80.2% yield) as a white solid. The NMR spectrum matched the literature spectrum. The purity of 2 was 99.1% by achiral HPLC analysis and had an optical purity of 100% by chiral HPLC analysis.

### Example—Preparation of 2 in Toluene

### Reaction Solution A (Compound 3 in Aqueous Basic Solution):

A 50 mL 3-necked round bottomed flask equipped with a water-cooled condenser, magnetic stir bar and thermometer was charged with 3.0 g of 4. To this solid was added 3N Hydrochloric acid (14 mL). Stirring was initiated and the resultant suspension was heated to 80° C. and maintained for 1 hr. The suspension slowly turned into a homogeneous, clear solution. The acidic solution was cooled to room temperature and washed with toluene (10 mL). The toluene layer was discarded. The aqueous layer was cooled to 10° C.), whereupon a 50% aqueous sodium hydroxide solution (35.0 g, 4.7 eq) was slowly added. The pH of the 3 in solution was adjusted between 7 to 9 and this solution was ready to be used in subsequent conversion as Reaction Solution A.

### Reaction Solution B (5 Triethylammonium Salt in Toluene):

In a separate vessel, 5 (4.5 g, 1.17 eq) was suspended in toluene (20 mL) and stirred until the acid dissolved. To this solution was added triethylamine (1.56 g, 1.4 eq). Stirring was continued for 15 minutes affording a clear, colorless solution. This solution of 5 triethylammonium salt in toluene was ready to be used in subsequent conversion as Reaction Solution B.

### The Condensation Reaction:

To Reaction Solution B was added DMTMM (4-(4,6-Dimethoxy-1,3,5-triazin-2-yl)-4-methylmorpholinium chloride) (3.96 g, 1.07 eq after correction for residual water). The stirring was continued, and the toluene slurry was heated to 35° C. affording a free-flowing slurry. To this heated slurry was added Reaction Solution A and the temperature was raised to 45-50° C. and stirred for two hours. HPLC analysis (achiral HPLC method) showed the reaction to be complete. The reaction slurry was diluted with heptane (30 mL) and stirred for 30 minutes, filtered and the filter cake was air dried, affording crude 2 (4.7 g) as a water wet white solid. The wet solid was dissolved in dichloromethane (20 mL), dried with sodium sulfate and clarified. To the stirred dichloromethane solution was added heptane (50 mL) and the resulting slurry was cooled to 5° C. and stirred for 30 minutes. The solid 2 was collected by filtration and dried under reduced pressure affording 2 (3.70 g, 72% yield). The NMR spectrum matched the literature spectrum. The purity of 2 was 99.8% by achiral HPLC analysis and had an optical purity of 100% by chiral HPLC analysis

### Example—Preparation of 2 in Dichloromethane (DCM)

### Reaction Solution A (Compound 3 in Aqueous Basic Solution):

A 50 mL 3-necked round bottomed flask equipped with a water-cooled condenser, magnetic stir bar and thermometer was charged with 4 (3.0 g). To this solid was added 3N Hydrochloric acid (14 mL). Stirring was initiated and the resultant suspension was heated to 80° C. and maintained for 1 hr. The suspension slowly turned into a homogeneous, clear solution. The acidic solution was cooled to room temperature and washed with toluene (10 mL). The toluene layer was discarded. The aqueous layer was cooled to 10° C., whereupon a 50% aqueous sodium hydroxide solution (35.0 g, 4.7 eq) was slowly added. The pH of the 3 in solution was adjusted between 7 to 9 and this solution was ready to be used in subsequent conversion as Reaction Solution A.

### Reaction Solution B (5 Triethylammonium Salt in DCM):

In a separate vessel, 5 (4.5 g, 1.17 eq) was dissolved in DCM (20 mL) and stirred until the acid dissolved. To this solution was added triethylamine (1.56 g, 1.4 eq). Stirring was continued for 15 minutes affording a clear, colorless solution. This solution of 5 in DCM was ready to be used in subsequent conversion as Reaction Solution B.

### The Condensation Reaction:

To Reaction Solution B was added DMTMM (4-(4,6-Dimethoxy-1,3,5-triazin-2-yl)-4-methylmorpholinium chloride) (3.96 g, 1.07 eq after correction for residual water). The stirring was continued, and the DCM solution was heated to 30° C. affording a nearly clear solution. To this stirred solution was added Reaction Solution A and the temperature was held at 30-35° C. and stirred for two hours. HPLC analysis (achiral HPLC method) showed the reaction to be complete. The reaction was diluted with DCM (20 mL) and the layers were allowed separate. The aqueous layer was discarded, and the DCM solution was washed with water (2×25 mL). The DCM solution was dried over sodium sulfate and clarified. The DCM solution was concentrated to a slurry and to this slurry was added heptane (40 mL). The slurry was cooled to 5° C. and stirred for 30 minutes. The solid 2 was collected by filtration and dried under reduced pressure affording purified 2 (4.62 g, 90.5% yield). The NMR spectrum matched the literature spectrum. The purity of 2 was 99.6% by achiral HPLC analysis and had an optical purity of 100% by chiral HPLC analysis.

### Example—Preparation of 2 using 2-chloro-4,6-dimethoxy-1,3,5-triazine (CDMT)

### Reaction Solution A (Compound 3 in Aqueous Basic Solution):

A 50 mL 3-necked round bottomed flask equipped with a water-cooled condenser, magnetic stir bar and thermometer was charged with 4 (3.0 g). To this solid was added 3N Hydrochloric acid (14 mL). Stirring was initiated and the resultant suspension was heated to 80° C. and maintained for 1 hr. The suspension slowly turned into a homogeneous, clear solution. The acidic solution was cooled to room temperature and washed with toluene (10 mL). The toluene layer was discarded. The aqueous layer was cooled to 10° C.), whereupon a 50% aqueous sodium hydroxide solution (35.0 g, 4.7 eq) was slowly added. The pH of the 3 in solution was adjusted between 7 to 9 and this solution was ready to be used in subsequent conversion.

### Reaction Solution B (5 Triethylammonium Salt in MTBE)

In a separate vessel, 5 (4.5 g, 1.17 eq) was added to MTBE (20 mL) and stirred until the acid dissolved. To this solution was added triethylamine (1.56 g, 1.4 eq). affording a clear, colorless solution. This solution of 5 triethylammonium salt in MTBE was ready to be used in subsequent conversion as Reaction Solution B

### The Condensation Reaction:

To the stirred Reaction Solution B was added CDMT (3.9 g, 2.0 eq) The stirring was continued, and the slurry was heated to 35° C. To this heated suspension was added Reaction Solution A and the temperature was raised to 45-50° C. and stirred for three hours. HPLC analysis (achiral HPLC method) showed about 30% conversion to 2 with a large portion of 3 and CDMT remaining. The batch was heated overnight and then assayed by HPLC. The assay was 45% of 2. The batch was cooled to room temperature and the layers were separated. The remaining MTBE layer was washed with 1 N HCl (3×100 mL) followed by saturated aqueous sodium bicarbonate solution (100 mL) and dried over sodium sulfate. The MTBE solution was concentrated to dryness to afford an oil. This oil was chromatographed on silica gel (using a gradient of 10% heptane in dichloromethane to 50% heptane in dichloromethane) and the fractions with 2 were pooled and concentrated to dryness affording 1.8 g of 2 (35.2% yield) as a white solid. The NMR spectrum matched the literature spectrum. The purity of 2 was 93.8% by achiral HPLC analysis and had an optical purity of 100% by chiral HPLC analysis.

### Example—Preparation of 2 Using EDCI and HOBT in N,N-dimethylformamide

A solution of 5 (10 g, 0.029 mol) in DMF (100 mL) was cooled to 5 to 10° C. To the cold solution were added triethylamine (3 g), HOBt hydrate (4.2 g, 1.07 eq)) and EDCl hydrochloride (7.9 g, 1.75 eq.) and stirred. To the above reaction mixture was added dextroamphetamine free base (isolated from the hydrolysis of 6 g of 4 and concentration of the MTBE extracts) and stirred at room temperature overnight. To the reaction mixture was added water (250 mL) and isopropyl acetate (250 mL), stirred for 3 h and separated. The aqueous layer was extracted with isopropyl acetate (200 mL) acetate. The combined organic layer was washed with 10% sodium carbonate solution (250 mL), 1 N hydrochloric acid (2×100 mL), water (100 mL) and then with saturated sodium chloride solution (100 mL).

The remaining organic solution was concentrated under reduced pressure to afford crude 2 white solid. The crude 2 was recrystallized using the MTBE/heptane method affording purified 2 (8.40 g, 81.4% yield) as a white solid. The NMR spectrum matched the literature spectrum. The purity of 2 was 99.1% by achiral HPLC analysis and had an optical purity of 100% by chiral HPLC analysis.

### Example—Telescoping the Process to 1 from L-Lysine HCl

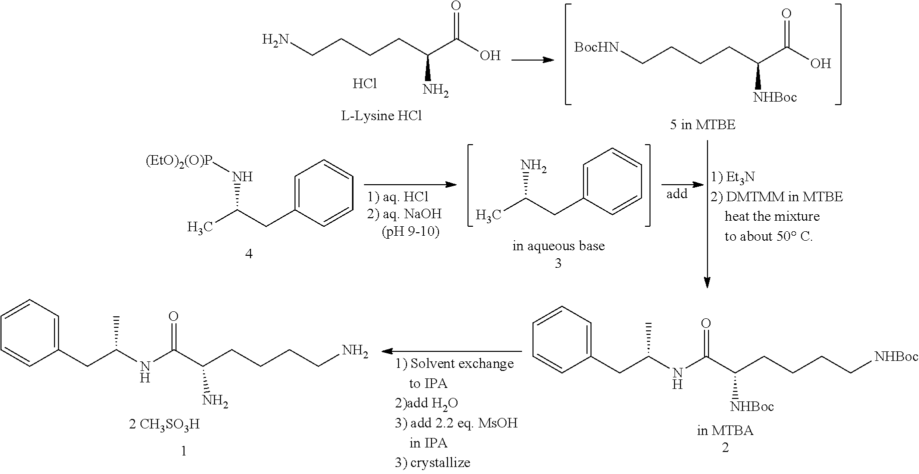

Lisdexamfetamine dimesylate
   Step 1: Preparation of 5 from L-Lysine HCl (Dioxane Free Process)

To a water cooled, stirred solution of L-lysine monohydrochloride (100 g,) in water (1 L) and acetonitrile (100 ml) was added solid sodium hydroxide (66 g, 3 eq) followed by di-tertiary butyl dicarbonate (280 g, 2.3 eq). The temperature of the stirred solution was raised to 55° C. and held overnight. The reaction mixture was cooled to room temperature and washed with toluene (2×100 mL), and these toluene washes were discarded. To the remaining aqueous solution was added MTBE (500 mL) and stirred biphasic mixture cooled to 10° C. and the pH of the solution was adjusted 6-7 by slowly adding 4 N HCl (200 mL). When the foaming ceased, the layers were separated, and the MTBE was discarded. The aqueous layer was returned to the flask and fresh MTBE (200 mL) was added. The stirred biphasic mixture was cooled to 15-20° C. and the pH was then adjusted to 2-3 by the slow addition of 4 N HCl (200 mL). The biphasic solution was stirred for 1 hour and the layers were allowed to settle for 1 hour and separated. The separated aqueous layer was back extracted with MTBE (100 mL). The combined MTBE organic layers were washed with water (2×100 mL) and the water washes were discarded. The remaining organic phase was dried (MgSO4), filtered and concentrated under reduced pressure to afford 5 (175 g, 93% yield but it still contains some MTBE by proton NMR). The product was diluted in MTBE to afford Reaction Solution A.

### Step 2: Conversion of 5 to 2.

Part 1: A suspension of 4 (50 g, 184 mmol) in 3N HCl (220 mL) was stirred and heated to 80° C. The reaction was monitored by HPLC. The suspension slowly turned into a homogeneous, clear solution. The acidic solution was cooled to room temperature and washed with toluene (2×200 mL). The toluene layers were discarded.

The aqueous phase was cooled to 10° C., stirred and treated with aqueous sodium hydroxide (39.5 g, 660.8 mmol in 37.5 mL water). The pH of the resulting solution was between 9 to 10. The solution is ready for the next step as Reaction Solution B.

Part 2: To the Reaction Solution A was added triethylamine (57 mL) and to this solution was added solid DMTMM in one portion. To this slurry was added Reaction Solution B and the biphasic mixture was heated to 50° C. The biphasic mixture was stirred for 2 h. The reaction was monitored by HPLC until the conversion was complete. To the biphasic mixture was added water (250 mL) and MTBE (250 mL). The 50° C. mixture was stirred for 10 min, and the stirrer was stopped, and the layers were separated. The remaining heated organic layer was washed with 1 N HCl (2×300 mL). The acidic washes were removed and discarded. The heated organic solution washed by saturated aqueous sodium bicarbonate solution (2×250 mL) and water (2×250 mL) and all of the aqueous washes were disposed. This MTBE solution of 2 is ready for the next step as Reaction Solution C.

### Step 3: Conversion of 2 to 1.

Reaction Solution C was charged to a flask equipped with an overhead stirrer and a distillation head with a water-cooled condenser, and was heated to 60° C. The MTBE distilled from the batch and was replaced with isopropanol (50 mL portions). When the distillation reached 80° C., the distillation was stopped, and the volume was adjusted to 300 mL by adding additional isopropanol. To this solution was charged water (12.5 mL) and methanesulfonic acid (15.5 mL, 240.0 mmol). The solution was heated to 80° C. for about 6 hours. The reaction was monitored by HPLC and after complete conversion, isopropyl acetate (175 mL) was added to induce crystallization. The stirred slurry was allowed to cool to room temperature.

The slurry was filtered, and the reaction flask and filter cake were washed with a chilled (5-10° C.) solution 30% isopropanol in isopropyl acetate (200 mL). The filter cake was dried in vacuo (at 30° C.) for 2 hours affording 1 as a white solid (40.5 g, 82.8% yield from L-Lysine hydrochloride).

### Example—Preparation of Optically Active 1 from Racemic 9

### Preparation of the 1:1 Mixture of 2 and 7

### Reaction Solution A:

A 1 L round bottomed flask equipped with a jacketed water condenser, mechanical stirrer and temperature probe was charged with 9 (75.0 g). To this solid was added 3N Hydrochloric acid (350 mL). Stirring was initiated and the resultant suspension was heated to 80° C. and maintained for 2 hr. The suspension slowly turned into a homogeneous, clear solution. The acidic solution was cooled to room temperature and washed with toluene (4×200 mL). The toluene layers were discarded. The aqueous layer was cooled to 10° C. at which point a 50% aqueous solution of sodium hydroxide (about 52.0 g,) was slowly added. The pH of the solution was adjusted between 7 to 9 and this solution of 8 in aqueous base was ready to be used in the subsequent conversion as Reaction Solution A.

### Reaction Solution B:

In a separate vessel, 5 (113.0 g, 1.17 eq) was added to MTBE (375 mL) and to this suspension was added triethylamine (39.2 g, 1.4 eq). Stirring was initiated and maintained for 30 minutes affording a clear, colorless solution. The solution is ready for the next step as Reaction Solution B.

### The Condensation Reaction:

To a 4 L, 3-necked round bottomed flask equipped with a mechanical stirrer, water cooled condenser, temperature probe and addition funnel was charged DMTMM (4-(4,6-Dimethoxy-1,3,5-triazin-2-yl)-4-methylmorpholinium chloride) (100.0 g, 1.1 eq after correction for residual water) followed by MTBE (1150 mL). The stirrer was started, and the suspension was heated to 35° C. affording a clear solution. To this solution was added Reaction Solution B and the mixture was stirred for about 30 minutes. To this heated solution was added Reaction Solution A over 30 minutes and the agitation was increased to efficiently mix the biphasic mixture. The temperature was raised to 45-50° C. and stirred for two hours. The pH of the aqueous layer of the biphasic solution was ˜7 and achiral and chiral HPLC analyses showed the reaction was complete. The reaction mixture was diluted with water (375 mL) and MTBE (375 mL) to dissolve any solids that separated. The reaction mixture temperature was maintained at 50° C. affording a clear biphasic mixture. The stirrer was stopped, and the aqueous layer was removed. The organic layer (still at 50° C.) was washed with water (750 mL). The aqueous layer was removed and the organic layer (still at 50° C.) was washed with a 1% aqueous solution of citric acid solution (750 mL). The aqueous layer was separated, and the organic layer (still at 50° C.) was washed with an aqueous solution of 2% NaHCO₃ (750 mL) followed by water washes (2×400 mL).

The remaining organic layer was concentrated under reduced pressure to about one-third of the original volume (removed ˜600 mL of MTBE) The remaining slurry was cooled to room temperature and heptanes (500 mL) was added and mechanically stirred for 5 h. The batch was cooled (about 5° C.) for 30 min and subsequently filtered. The collected solid was washed with a mixture of 30% MTBE/heptane (250 mL total) and dried under vacuum affording 121.0 g (94% yield) of the 1:1 mixture 2 and 7 as a white, crystalline solid. The NMR spectrum was consistent with the mixture. The purity of the 2 and 7 mixture was 99.6% by achiral HPLC (single peak) and was 51.3% 2 and 48.7% 7 by the chiral HPLC analysis.

Preparation of Purified 1 from the Mixture of 2 and 7:

To a 3 L, 3-necked round bottomed flask, fitted with a water-cooled reflux condenser, temperature probe and mechanical stirrer was charged the 1:1 mixture of 2 and 7 (150 g) followed by a mixture of isopropanol (527 mL) and water (40.5 mL). The stirrer was started and methanesulfonic acid (46.2 mL; 2.2 equivalents) was added. The solution was heated to 80° C. and held at that temperature for 12 hours. At this point the achiral HPLC analysis indicated the reaction was complete as a mixture of 1 (51%) and 6 (49%). Isopropyl acetate (1.03 L) was added over 30 minutes, while maintaining the batch temperature at 80° C. The solution was cooled to 55° C. and to the hazy solution was added isopropanol (75 mL) and water (2.2 mL) to dissolve the solids. The solution was cooled to ambient temperature and stirred overnight. The solids were collected by filtration and the filter cake was washed with 30% isopropanol in isopropyl acetate (2×100 mL) followed by isopropyl acetate (100 mL). The solid was vacuum dried at 40° C. affording 61.5 g of white solid material (41.7% isolated yield from the 1:1 mixture charged in this step). The achiral HPLC analysis indicated that the solid was a mixture of 6.5% 6 (rt=6.44 min) and 93.5% 1 (rt=7.93 min).

To a 3 L, 3-necked round bottomed flask, fitted with a water-cooled reflux condenser, temperature probe and mechanical stirrer was charged the above solid (60 g) and isopropanol (1.2 L). The mixture was heated to 80° C. and water was added to achieve a complete solution (15 mL). Isopropyl acetate (1.8 L) was added over a period of 1 hour, while maintaining the batch temperature of 80° C. The stirred mixture was cooled to ambient temperature and stirred for 2 hours. The solids were collected by filtration and washed with isopropyl acetate (2×100 mL). The solid filter cake was vacuum dried at 40° C. to afford 54.5 g of a white solid (91% recovery). The achiral HPLC analysis indicated that the solid was a mixture of 0.82% 6 (rt=6.44 min) and 99.18% 1 (rt=7.93 min).

A second recrystallization of the above material afforded a 92% recovery of purified 1 which was >99.9% 1 and <0.1% of 6 (34.9% yield from the 150 g of the 1:1 mixture of 2 and 7).

This material matched the literature spectroscopic data for Lisdexamfetamine dimesylate.

### Definitions

The term “crystal” refers to a form of a solid state of matter, which is distinct from its amorphous solid state. Crystals display characteristic features including a lattice structure, characteristic shapes and optical properties such as refractive index. A crystal consists of atoms arranged in a pattern that repeats periodically in three dimensions.

An “anhydrous crystal form” lacks bound water molecules.

The term “polymorph” refers to crystallographically distinct forms of a substance.

The term “amine” refers to a —NH2 group.

The term “DMTMM” refers to 4-(4,6-Dimethoxy-1,3,5-triazin-2-yl)-4-methylmorpholinium chloride, which is an organic triazine derivative commonly used for activation of carboxylic acids, particularly for amide synthesis. DMTMM may be illustrated having the following structure

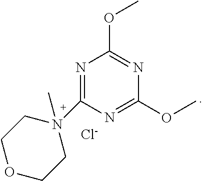

The compound naming may include without limitation the following:
Compound 5: BOC-L-Lys(BOC)—OH;

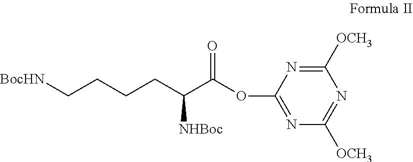 [SMILES: COc1nc(OC)nc(OC(=O)[C@H](CCCCNC(=O)OC(C)(C)C)NC(=O)OC(C)(C)C)n1]

Compound [5a]: BOC-L-Lys(BOC)—O-DMT Formula II;
Compound 4: d-diethyl(1-phenylpropan-2-yl) phosphoramidate;
Compound 9: racemic d-, l-diethyl(1-phenylpropan-2-yl) phosphoramidate;
Compound 3: dextroamphetamine

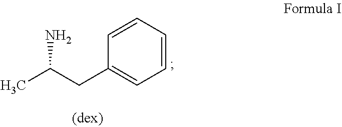

Compound 8: d-, l-amphetamine

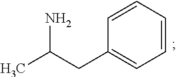

Compound 2: BOC-L-Lys(BOC)-dextroamphetamine

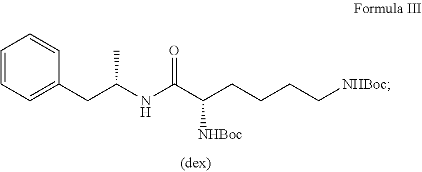 [SMILES: C[C@@H](Cc1ccccc1)NC(=O)[C@H](CCCCNC(=O)OC(C)(C)C)NC(=O)OC(C)(C)C]

Compound 7: BOC-L-Lys(BOC)-levoamphetamine

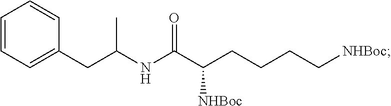 [SMILES: CC(Cc1ccccc1)NC(=O)[C@H](CCCCNC(=O)OC(C)(C)C)NC(=O)OC(C)(C)C]

Compound [1a]: deprotected L-lysine amphetamine amide salt;

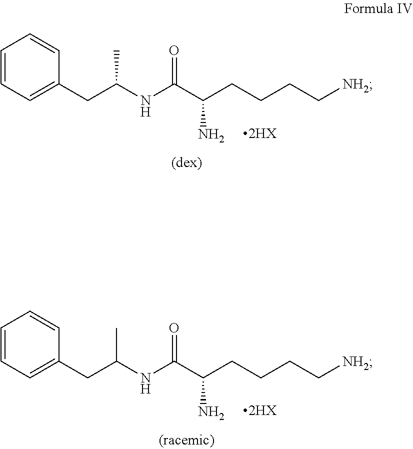 [SMILES: C[C@@H](Cc1ccccc1)NC(=O)[C@@H](N)CCCCN ; CC(Cc1ccccc1)NC(=O)[C@@H](N)CCCCN]

Compound 1=Lisdexamfetamine dimesylate (LDX);
Compound 6=Lislevoamfetamine dimesylate (LLX).

The D10, D50 and D90 represent the 10th percentile, median or the 50th percentile and the 90th percentile of the particle size distribution, respectively, as measured by diameter. That is, the D10 (D50, D90) is a value on the distribution such that 10% (50%, 90%) of the particles have a volume of this value or less.

An “effective amount of drug” is an amount of lisdexamfetamine dimesylate which is effective to treat or prevent a condition in a living organism to whom it is administered over some period of time, e.g., provides a therapeutic effect during a desired dosing interval.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

A composition refers broadly to any composition containing one or more amphetamine prodrugs. The composition can comprise a dry formulation, an aqueous solution, or a sterile composition. Compositions comprising the compounds described herein may be stored in freeze-dried form and may be associated with a stabilizing agent such as a carbohydrate. In use, the composition may be deployed in an aqueous solution containing salts, e.g., NaCl, detergents such as sodium dodecyl sulfate (SDS), and other components.

In one embodiment, the amphetamine prodrug itself exhibits a sustained release profile. Thus, the invention provides a pharmaceutical composition exhibiting a sustained release profile due to the amphetamine prodrug.

In another embodiment, a sustained release profile is enhanced or achieved by including a hydrophilic polymer in the pharmaceutical composition. Suitable hydrophilic polymers include, but are not limited to, natural or partially or totally synthetic hydrophilic gums such as acacia, gum tragacanth, locust bean gum, guar gum, and karaya gum; cellulose derivatives such as methyl cellulose, hydroxymethyl cellulose, hydroxypropylmethyl cellulose, hydroxypropyl cellulose, hydroxyethyl cellulose, and carboxymethyl cellulose; proteinaceous substances such as agar, pectin, carrageen, and alginates; hydrophilic polymers such as carboxypolymethylene; gelatin; casein; zein; bentonite; magnesium aluminum silicate; polysaccharides; modified starch derivatives; and other hydrophilic polymers known in the art. Preferably, the hydrophilic polymer forms a gel that dissolves slowly in aqueous acidic media thereby allowing the amphetamine prodrug to diffuse from the gel in the stomach. Then when the gel reaches the higher pH medium of the intestines, the hydrophilic polymer dissolves in controlled quantities to allow further sustained release. Preferred hydrophilic polymers are hydroxypropyl methylcelluloses such as Methocel ethers, e.g., Methocel E10M® (Dow Chemical Company, Midland, Mich.). One of ordinary skill in the art would recognize a variety of structures, such as bead constructions and coatings, useful for achieving particular release profiles. See, e.g., U.S. Pat. No. 6,913,768.

In addition to the amphetamine prodrug, the pharmaceutical compositions of the invention further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, and other pharmaceutical additives known in the art. For example, in a preferred embodiment, the pharmaceutical composition comprises magnesium stearate. In another preferred embodiment, the pharmaceutical composition comprises microcrystalline cellulose (e.g., Avicel® PH-102), croscarmellose sodium, and magnesium stearate. See, e.g., Table 62.

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc. A preferred diluent is microcrystalline cellulose (e.g., Avicel® PH-102). Preferred ranges for the amount of diluent by weight percent include about 40% to about 90%, about 50% to about 85%, about 55% to about 80%, about 50% to about 60%, and increments therein.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate.

For tablet dosage forms, the pharmaceutical composition is subjected to pressure from a punch and dye. Among other purposes, a lubricant can help prevent the composition from sticking to the punch and dye surfaces. A lubricant can also be used in the coating of a coated dosage form. Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil. A preferred lubricant is magnesium stearate. The amount of lubricant by weight percent is preferably less than about 5%, more preferably 4%, 3%, 2%, 1.5%, 1%, or 0.5%, or increments therein.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include both hydrophobic and hydrophilic plasticizers such as, but not limited to, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets. In one embodiment, the plasticizer facilitates the release of the amphetamine prodrug from the dosage form.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®). Preferred disintegrants include croscarmellose sodium and microcrystalline cellulose (e.g., Avicel® PH-102). Preferred ranges for the amount of disintegrant by weight percent include about 1% to about 10%, about 1% to about 5%, about 2% to about 3%, and increments therein.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; methylene chloride; vegetable oil; polyethylene glycol; propylene glycol; and glycerin.

The pharmaceutical composition can comprise a buffer. Buffers include, but are not limited to, lactic acid, citric acid, acetic acid, sodium lactate, sodium citrate, and sodium acetate.

Any pharmaceutically acceptable colorant can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks. Preferred colors for gelatin capsules include white, medium orange, and light blue.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

The pharmaceutical compositions of the invention can also include one or more preservatives and/or stabilizers to improve storagability. These include, but are not limited to, alcohol, sodium benzoate, butylated hydroxy toluene, butylated hydroxyanisole, and ethylenediamine tetraacetic acid.

Other pharmaceutical additives include gelling agents such as colloidal clays; thickening agents such as gum tragacanth and sodium alginate; wetting agents such as lecithin, polysorbates, and laurylsulphates; humectants; antioxidants such as vitamin E, caronene, and BHT; adsorbents; effervescing agents; emulsifying agents, viscosity enhancing agents; surface active agents such as sodium lauryl sulfate, dioctyl sodium sulfosuccinate, triethanolamine, polyoxyethylene sorbitan, poloxalkol, and quaternary ammonium salts; and other miscellaneous excipients such as lactose, mannitol, glucose, fructose, xylose, galactose, sucrose, maltose, xylitol, sorbitol, chloride, sulfate and phosphate salts of potassium, sodium, and magnesium.

The pharmaceutical compositions can be manufactured according to any method known to those of skill in the art of pharmaceutical manufacture such as, for example, wet granulation, dry granulation, encapsulation, direct compression, slugging, etc. For instance, a pharmaceutical composition can be prepared by mixing the amphetamine prodrug with one or more pharmaceutical additives with an aliquot of liquid, preferably water, to form a wet granulation. The wet granulation can be dried to obtain granules. The resulting granulation can be milled, screened, and blended with various pharmaceutical additives such as water-insoluble polymers and additional hydrophilic polymers. In one embodiment, an amphetamine prodrug is mixed with a hydrophilic polymer and an aliquot of water, then dried to obtain granules of amphetamine prodrug encapsulated by hydrophilic polymer.

After granulation, the pharmaceutical composition is preferably encapsulated, e.g., in a gelatin capsule. The gelatin capsule can contain, for example, kosher gelatin, titanium dioxide, and optional colorants. Alternatively, the pharmaceutical composition can be tableted, e.g., compressed and optionally coated with a protective coating that dissolves or disperses in gastric juices.

The pharmaceutical compositions of the invention can be administered by a variety of dosage forms. Any biologically-acceptable dosage form known in the art, and combinations thereof, are contemplated. Examples of preferred dosage forms include, without limitation, tablets including chewable tablets, film-coated tablets, quick dissolve tablets, effervescent tablets, multi-layer tablets, and bi-layer tablets; caplets; powders including reconstitutable powders; granules; dispersible granules; particles; microparticles; capsules including soft and hard gelatin capsules; lozenges; chewable lozenges; cachets; beads; liquids; solutions; suspensions; emulsions; elixirs; and syrups.

The pharmaceutical composition is preferably administered orally. Oral administration permits the maximum release of amphetamine, provides sustained release of amphetamine, and maintains abuse resistance. Preferably, the amphetamine prodrug releases the amphetamine over a more extended period of time as compared to administering unbound amphetamine.

Oral dosage forms can be presented as discrete units, such as capsules, caplets, or tablets. In a preferred embodiment, the invention provides a solid oral dosage form comprising an amphetamine prodrug that is smaller in size compared to a solid oral dosage form containing a therapeutically equivalent amount of unbound amphetamine. In one embodiment, the oral dosage form comprises a gelatin capsule of size 2, size 3, or smaller (e.g., size 4). The smaller size of the amphetamine prodrug dosage forms promotes ease of swallowing.

Soft gel or soft gelatin capsules may be prepared, for example, by dispersing the formulation in an appropriate vehicle (e.g., vegetable oil) to form a high viscosity mixture. This mixture then is encapsulated with a gelatin-based film. The industrial units so formed are then dried to a constant weight.

Chewable tablets can be prepared by mixing the amphetamine prodrug with excipients designed to form a relatively soft, flavored tablet dosage form that is intended to be chewed.

Conventional tablet machinery and procedures (e.g., direct compression, granulation, and slugging) can be utilized.

Film-coated tablets can be prepared by coating tablets using techniques such as rotating pan coating methods and air suspension methods to deposit a contiguous film layer on a tablet.

Compressed tablets can be prepared by mixing the amphetamine prodrug with excipients that add binding qualities. The mixture can be directly compressed, or it can be granulated and then compressed.

The pharmaceutical compositions of the invention can alternatively be formulated into a liquid dosage form, such as a solution or suspension in an aqueous or non-aqueous liquid.

The liquid dosage form can be an emulsion, such as an oil-in-water liquid emulsion or a water-in-oil liquid emulsion. The oils can be administered by adding the purified and sterilized liquids to a prepared enteral formula, which then is placed in the feeding tube of a patient who is unable to swallow.

For oral administration, fine powders or granules containing diluting, dispersing, and/or surface-active agents can be presented in a draught, in water or a syrup, in capsules or sachets in the dry state, in a non-aqueous suspension wherein suspending agents may be included, or in a suspension in water or a syrup. Liquid dispersions for oral administration can be syrups, emulsions, or suspensions. The syrups, emulsions, or suspensions can contain a carrier, for example, a natural gum, agar, sodium alginate, pectin, methylcellulose, carboxymethylcellulose, saccharose, saccharose with glycerol, mannitol, sorbitol, and polyvinyl alcohol.

The dose range of the amphetamine prodrug for humans will depend on a number of factors including the age, weight, and condition of the patient. Tablets and other dosage forms provided in discrete units can contain a daily dose, or an appropriate fraction thereof, of one or more amphetamine prodrugs. The dosage form can contain a dose of about 2.5 mg to about 500 mg, about 10 mg to about 250 mg, about 10 mg to about 100 mg, about 25 mg to about 75 mg, or increments therein of one or more of the amphetamine prodrugs. In a preferred embodiment, the dosage form contains 30 mg, 50 mg, or 70 mg of an amphetamine prodrug.

The dosage form can utilize any one or any combination of known release profiles including, but not limited to immediate release, extended release, pulse release, variable release, controlled release, timed release, sustained release, delayed release, and long acting.

The pharmaceutical compositions of the invention can be administered in a partial, i.e., fractional dose, one or more times during a 24-hour period. Fractional, single, double, or other multiple doses can be taken simultaneously or at different times during a 24-hour period. The doses can be uneven doses with regard to one another or with regard to the individual components at different administration times. Preferably, a single dose is administered once daily. The dose can be administered in a fed or fasted state.

The dosage units of the pharmaceutical composition can be packaged according to market need, for example, as unit doses, rolls, bulk bottles, blister packs, and so forth. The pharmaceutical package, e.g., blister pack, can further include or be accompanied by indicia allowing individuals to identify the identity of the pharmaceutical composition, the prescribed indication (e.g., ADHD), and/or the time periods (e.g., time of day, day of the week, etc.) for administration. The blister pack or other pharmaceutical package can also include a second pharmaceutical product for combination therapy.

It will be appreciated that the pharmacological activity of the compositions of the invention can be demonstrated using standard pharmacological models that are known in the art.

Furthermore, it will be appreciated that the inventive compositions can be incorporated or encapsulated in a suitable polymer matrix or membrane for site-specific delivery, or can be functionalized with specific targeting agents capable of effecting site specific delivery. These techniques, as well as other drug delivery techniques, are well known in the art.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention.

Accordingly, the scope of the invention is determined by the scope of the following claims and their equitable Equivalents.

1. (canceled)

2. The process according to claim 6, wherein the amphetamine is dextroamphetamine, levoamphetamine, or a racemic mixture (d-, l-amphetamine).

3. The process according to claim 6, wherein the amphetamine is dextroamphetamine.

4. The process according to claim 6, wherein the BOC-L-Lys(BOC)—N-amphetamine is BOC-L-Lys(BOC)—N-dextroamphetamine, BOC-L-Lys(BOC)—N-levoamphetamine, or a racemic mixture (BOC-L-Lys(BOC)—N-(d-,l-)amphetamine.

5. The process according to claim 6, wherein the BOC-L-Lys(BOC)—N-amphetamine is BOC-L-Lys(BOC)—N-dextroamphetamine.

6. A process for making a lysine-amphetamine compound, comprising the steps: (i) reacting a protected L-Lysine ammonium salt BOC-L-Lys(BOC)—O—X—NH+ in an organic solvent with an amphetamine in aqueous base in the presence of a coupling agent to form a biphasic mixture and heating the biphasic mixture at 30-50° C. to yield BOC-L-Lys(BOC)—N-amphetamine and then (ii) reacting under appropriate conditions the BOC-L-Lys(BOC)—N-amphetamine with methanesulfonic acid (MsOH), and crystallizing the product to obtain lisdexamfetamine dimesylate, wherein the protected L-Lysine ammonium salt BOC-L-Lys(BOC)—O—X—NH+ is prepared under chemically sufficient conditions using an amine base X selected from the group consisting of triethylamine, N,N-diisopropylethylamine amine, 1,4-diazabicyclo[2.2.2]octane (DABCO) or N-methyl morpholine.

7. The process according to claim 6, wherein the protected L-Lysine ammonium salt BOC-L-Lys(BOC)—O—X—NH+ is prepared under chemically sufficient conditions where X is triethylamine to form BOC-L-Lys(BOC)—O Et₃NH+.

8. The process according to claim 6, wherein the BOC-L-Lys(BOC)—O—X—NH+ is prepared by reacting BOC-L-Lys(BOC)—OH with an amine base where X is triethylamine (Et₃N) in methyl tert butyl ether (MTBE) to obtain BOC-L-Lys(BOC)—O Et₃NH+.

9. The process according to claim 6, wherein the coupling agent is DMTMM ((4-(4,6-dimethoxy-1,3,5-triazin-2-yl)-4-methyl-morpholinium chloride).

10. The process according to claim 6, wherein the coupling agent is selected from the group consisting of: 2-chloro-4,6-dimethoxy-1,3,5-triazine (CDMT) or 4-(4,6-Dimethoxy-1,3,5-triazin-2-yl)-4-methylmorpholin-4-ium chloride (DMTMM) or 4-(4,6-Dimethoxy-1,3,5-triazin-2-yl)-4-methylmorpholin-4-ium tosylate (DMTMMOTs), 4-(4,6-Dimethoxy-1,3,5-triazin-2-yl)-4-methylmorpholin-4-ium tetrafluoroborate (DMTMMBF₄), 1-Ethyl-3-(3-dimethylaminopropyl)carbodiimide (EDCI)/1-hydroxybenzotriazole (HOBt), and a mixture thereof.

11. The process according to claim 6, wherein the organic solvent is water immiscible and is selected from the group consisting of: Methyl t-butyl ether (MTBE), N,N-dimethylformamide (DMF), dichloromethane (DCM), toluene, acetonitrile, ethyl acetate, isopropanol, isopropyl acetate, cyclohexane, methylene chloride, diethyl ether, tetrahydrofuran (THF), and a mixture thereof.

12. The process according to claim 6, wherein the dextroamphetamine is prepared by deprotecting diethoxy-phosphoramidate with aqueous HCl and NaOH to produce aqueous dexamphetamine ((2S)-1-phenylpropan-2-amine).

13. The process according to claim 6, wherein the amphetamine is a racemic mixture (d-, l-amphetamine) prepared by deprotecting a racemic mixture of (S—, R-)-diethoxy-phosphoramidate with aqueous HCl and NaOH to produce an aqueous racemic mixture of dexamphetamine ((2S)-1-phenylpropan-2-amine) and levoamphetamine ((2R)-1-phenylpropan-2-amine).

14. The process according to claim 6, where crystallizing is performed using a crystallization solution comprising isopropanol in isopropyl acetate, and after crystallization yields a 95:5 ratio (purity) of lisdexamfetamine dimesylate to lislevoamfetamine dimesylate.

15. The process according to claim 6, where crystallizing is performed using a crystallization solution comprising isopropanol in isopropyl acetate, and after a re-crystallization yields a 98.7:1.2 ratio (purity) of lisdexamfetamine dimesylate to lislevoamfetamine dimesylate.

16. The process according to claim 6, where crystallizing is performed using a crystallization solution comprising isopropanol in isopropyl acetate, and after a second re-crystallization yields a 99.9:<0.1 ratio (purity) of lisdexamfetamine dimesylate to lislevoamfetamine dimesylate

17. The process according to claim 16, wherein the isopropanol is selected from the group consisting of isopropanol (absolute), isopropanol (commercial 91% solution), n-propanol, and a mixture thereof.

18. A lysine-amphetamine compound made by the process of claim 6.

19. A method of preparing a lysine-amphetamine amide salt, comprising the steps of mixing an amphetamine compound of Formula I with a triazine activated protected L-lysine compound of Formula II under conditions to provide pure crystalline L-lysine-amphetamine amide of Formula III directly from the reaction mixture, where Formula I is represented by: 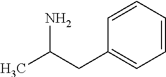
or a salt thereof;
and Formula II is represented by: 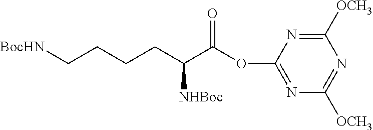 [SMILES: COc1nc(OC)nc(OC(=O)[C@H](CCCCNC(=O)OC(C)(C)C)NC(=O)OC(C)(C)C)n1]
and formula III is represented by: 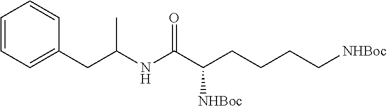 [SMILES: CC(Cc1ccccc1)NC(=O)[C@H](CCCCNC(=O)OC(C)(C)C)NC(=O)OC(C)(C)C]
and then acid deprotected to provide the L-lysine amphetamine amide salt of Formula IV

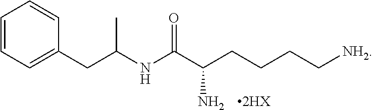

20. The process according to claim 19 where formula I is dextroamphetamine 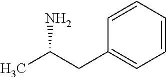
having a purity of at least 90% ee
and formula III is: 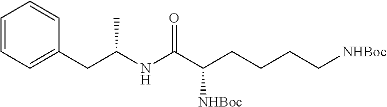 [SMILES: C[C@@H](Cc1ccccc1)NC(=O)[C@H](CCCCNC(=O)OC(C)(C)C)NC(=O)OC(C)(C)C]
and formula IV is:

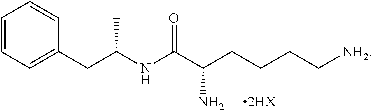

21. The process according to claim 20 where the source of dextroamphetamine is a salt or a phosphoramidate ester.

22. The process according to claim 21 where the dextroamphetamine salt is selected from the group consisting of: dextroamphetamine hydrochloride, dextroamphetamine sulfate, dextroamphetamine saccharate, dextroamphetamine tartrate, and a mixture thereof.

23. The process according to claim 22 where the dextroamphetamine salt is dextroamphetamine sulfate.

24. The process according to claim 23 where a heated organic solution of a carboxylate salt of the bis BOC protected L-Lysine is treated with an aqueous basic solution of dextroamphetamine to form an aqueous mixture and this aqueous mixture is then treated with an organic solution of a triazine coupling reagent in an organic solvent, and this aqueous-organic mixture is heated until the reaction is complete.

25. The process according to claim 24 where the triazine coupling reagent is 2-chloro-4,6-dimethoxy-1,3,5-triazine (CDMT) or 4-(4,6-Dimethoxy-1,3,5-triazin-2-yl)-4-methylmorpholin-4-ium chloride (DMTMM).

26. The process according to claim 25 where the triazine coupling reagent is DMTMM.

27. The process according to claim 26 where the DMTMM is prepared in a chilled organic reaction mixture from CDMT and n-methyl morpholine.

28. The process according to claim 24 where the carboxylate salt of the bis BOC protected L-lysine is selected from the group consisting of: a sodium salt, a potassium salt, a triethylammonium salt, and a mixture thereof.

29. The process according to claim 28 where the carboxylate salt of the bis BOC protected L-lysine is the triethylammonium salt.

30. The process according to claim 24 where the organic solvent is water immiscible and is selected from the group consisting of: cyclohexane, toluene, methylene chloride, diethyl ether, THF, Methyl t-butyl ether (MTBE), acetonitrile, ethyl, isopropyl acetate, and a mixture thereof.

31. The process according to claim 30 where the organic solvent is selected from the group consisting of: methylene chloride, MTBE, ethyl acetate, isopropyl acetate, and a mixture thereof.

32. The process according to claim 24 where the aqueous basic solution comprises a base is selected from the group consisting of: a sodium base, potassium base, lithium base, an amine base, and a mixture thereof.

33. The process according to claim 32 where the sodium base is selected from the group consisting of: sodium bicarbonate, sodium carbonate and sodium hydroxide.

34. The process according to claim 32 where the sodium base is sodium bicarbonate.

35. The process according to claim 32 where the potassium base is selected from the group consisting of: potassium bicarbonate, potassium carbonate and potassium hydroxide.

36. The process according to claim 32 where the potassium base is potassium bicarbonate.

37. The process according to claim 32 where the amine base is selected from the group consisting of: triethylamine, N,N-diisopropylethylamine, 1,4-diazabicyclo[2.2.2]octane (DABCO) and N-methyl morpholine.

38. The process according to claim 32 where the amine base is triethylamine.

39. The process according to claim 24 where the organic solvent is water miscible and is selected from the group consisting of methanol, ethanol, isopropanol, n-propanol, acetonitrile, THF, N,N-dimethylformamide (DMF), dimethyl sulfoxide, and a mixture thereof.

40. The process according to claim 24 where the organic solvent is water miscible and is selected from the group consisting of ethanol, isopropanol, n-propanol, THF, acetonitrile, and a mixture thereof.

41. The process according to claim 24 where the organic solvent is water miscible and is selected from ethanol, isopropanol, and a mixture thereof.

42. The process according to claim 19 where the L-lysine amphetamine amide salt of Formula IV is crystallized using a compound selected from the group consisting of isopropanol (absolute), isopropanol (commercial 91% solution), n-propanol, ethanol, N,N-dimethylformamide, and mixtures thereof.

43. The process according to claim 19 where the L-lysine amphetamine amide salt of Formula IV is crystallized using a compound selected from the group consisting of isopropanol (absolute), isopropanol (commercial 91% solution), n-propanol, ethanol, N,N-dimethylformamide, and mixtures thereof, and after crystallization yields a 95:5 ratio (purity) of lisdexamfetamine dimesylate to lislevoamfetamine dimesylate.

44. The process according to claim 42 wherein L-lysine amphetamine amide salt of Formula IV is re-crystallized and yields a 98.8:1.2 ratio (purity) of lisdexamfetamine dimesylate to lislevoamfetamine dimesylate.

45. The process according to claim 44 wherein L-lysine amphetamine amide salt of Formula IV is re-crystallized a second time and yields a 99.9: <0.1 ratio (purity) of lisdexamfetamine dimesylate to lislevoamfetamine dimesylate.

46. A process comprising: (i) reacting BOC-L-Lys(BOC)—OH with triethylamine (Et₃N) in methyl tert butyl ether (MTBE) to obtain BOC-L-Lys(BOC)—O Et₃NH+, and then (ii) deprotecting diethoxy-phosphoramidate with aqueous HCl and NaOH to produce aqueous dexamphetamine ((2S)-1-phenylpropan-2-amine), and then (iii) reacting BOC-L-Lys(BOC)—O Et₃NH-+ with dexamphetamine in the presence of DMTMM ((4-(4,6-dimethoxy-1,3,5-triazin-2-yl)-4-methyl-morpholinium chloride) and heating the biphasic mixture at 40-50° C. to couple the dexamphetamine with the protected lysine carboxylic acid and yield BOC-L-Lys(BOC)—O-dexamphetamine, and then (iv) reacting the BOC-L-Lys(BOC)—O-dexamphetamine with MsOH in isopropanol, and crystallizing to obtain lisdexamfetamine dimesylate.

47. A process comprising: (i) reacting BOC-L-Lys(BOC)—OH with triethylamine (Et₃N) in methyl tert butyl ether (MTBE) to obtain BOC-L-Lys(BOC)—O Et₃NH+, and then (ii) deprotecting a racemic mixture of (S—, R-)-diethoxy-phosphoramidate with aqueous HCl and NaOH to produce an aqueous racemic mixture of dexamphetamine ((2S)-1-phenylpropan-2-amine) and levoamphetamine ((2R)-1-phenylpropan-2-amine), and then (iii) reacting BOC-L-Lys(BOC)—O Et₃NH-+ with the aqueous racemic mixture of dexamphetamine and levoamphetamine in the presence of DMTMM ((4-(4,6-dimethoxy-1,3,5-triazin-2-yl)-4-methyl-morpholinium chloride) and heating the biphasic mixture at 40-50° C. to couple the dexamphetamine and levoamphetamine with the protected lysine carboxylic acid to yield a racemic mixture of BOC-L-Lys(BOC)—O-dexamphetamine and BOC-L-Lys(BOC)—O-levoamphetamine, and then (iv) reacting the racemic mixture of BOC-L-Lys(BOC)—O-dexamphetamine and BOC-L-Lys(BOC)—O-levoamphetamine with MsOH in isopropanol, and crystallizing to obtain a racemic mixture of lisdexamfetamine dimesylate and lislevoamfetamine dimesylate.

48. The process according to claim 46 wherein the process yields 85-90%.

49. The process according to claim 47 wherein the process yields 85-90%.

50. The process according to claim 46 wherein the process includes at least one re-crystallization step.

51. The process according to claim 47 wherein the process includes at least one re-crystallization step.

52. The process according to claim 47 wherein the racemic process after crystallization yields a 95:5 ratio (purity) of lisdexamfetamine dimesylate to lislevoamfetamine dimesylate.

53. The process according to claim 52 wherein the process after a first re-crystallization yields a 98.8:1.2 ratio (purity) of lisdexamfetamine dimesylate to lislevoamfetamine dimesylate.

54. The process according to claim 53 wherein the process after a second re-crystallization yields a 99.9: <0.1 ratio (purity) of lisdexamfetamine dimesylate to lislevoamfetamine dimesylate.

55. Crystalline lisdexamfetamine dimesylate having a purity ratio of (95:5) or (98.8:1.2) or (99.9: <0.1) of lisdexamfetamine dimesylate to lislevoamfetamine dimesylate.

56. A pharmaceutical composition comprising crystalline lisdexamfetamine dimesylate of claim 55 and a pharmaceutically acceptable carrier.

57. A method of treating attention deficit hyperactivity disorder in a patient in need thereof, comprising the steps of orally administering the pharmaceutical composition of claim 56.

58. A compound, DMT-amphetamine.

59. A mixture of two enantiomers with diastereomers of each comprising levoamphetamine-L-lysine, levoamphetamine-D-lysine, dexamphetamine-L-lysine, and dexamphetamine-D-lysine.

60. The mixture according to claim 58 prepared by mixing an aqueous amphetamine compound and directly coupling with a triazine activated protected lysine compound.

61. A fluidic process, comprising: preparing an aqueous solution of free base amphetamine, coupling the amphetamine with a BOC-L-Lys(BOC)—OH in MTBE and DMTMM in MTBE, heating the aqueous mixture to 50° C. to obtain a biphasic solution.
